(19) United States
(12) Patent Application Publication
(10) Pub. No.: US 2024/0225535 A1
(43) Pub. Date: Jul. 11, 2024

(54) SYSTEMS, DEVICES, AND METHODS RELATING TO MEDICATION DOSE GUIDANCE

(71) Applicant: ABBOTT DIABETES CARE INC., Alameda, CA (US)

(72) Inventors: Matthew T. Novak, Oakland, CA (US); Aparajita Bhattacharya, Dublin, CA (US); Gary A. Hayter, Oakland, CA (US)

(21) Appl. No.: 18/511,003

(22) Filed: Nov. 16, 2023

Related U.S. Application Data
(60) Provisional application No. 63/426,114, filed on Nov. 17, 2022.

(51) Int. Cl. A61B 5/00; A61B 5/145; G16H 50/70; G16H 70/40

(52) U.S. Cl. CPC A61B 5/4839; A61B 5/0022; A61B 5/14532; G16H 50/70; G16H 70/40

(57) ABSTRACT

Systems, devices and methods are provided for titration of a medication dose for a patient or user. The titration may be based on determining a hypoglycemic risk of a user for a plurality of time-of-day periods. The determination of the hypoglycemic risk may be accomplished by known methods, including the glucose pattern analysis, glucose dysregulation analysis, low alarm frequency analysis, and combinations thereof. The system may then select a recommended action based on the analyte pattern type. The system may then store an indicator of the recommended action in a computer memory for output. The system may then output the recommended action. The recommended action may be outputted to the user, an HCP, or a caregiver. The recommended action may also vary depending on who is receiving the recommended action.

### CROSS-REFERENCE TO RELATED APPLICATIONS

This application claims priority to U.S. Provisional Application No. 63/426,114, filed Nov. 17, 2022, which is herein expressly incorporated by reference in its entirety for all purposes.

### FIELD

The subject matter described herein relates generally to systems, devices, and methods relating to medication dose guidance such as, for example, the determination of an insulin dose for the treatment of elevated glucose levels resulting from diabetes.

### BACKGROUND

The detection and/or monitoring of analyte levels, such as glucose, ketones, lactate, oxygen, hemoglobin A1C, or the like, can be vitally important to the health of an individual having diabetes. Patients suffering from diabetes mellitus can experience complications including loss of consciousness, cardiovascular disease, retinopathy, neuropathy, and nephropathy. Diabetics are generally required to monitor their glucose levels to ensure that they are being maintained within a clinically safe range, and may also use this information to determine if and/or when insulin is needed to reduce glucose levels in their bodies or when additional glucose is needed to raise the level of glucose in their bodies.

Growing clinical data demonstrates a strong correlation between the frequency of glucose monitoring and glycemic control. Despite such correlation, many individuals diagnosed with a diabetic condition do not monitor their glucose levels as frequently as they should due to a combination of factors including inconvenience, testing discretion, pain associated with glucose testing, and cost.

For patients that rely on the administration of medications (e.g., insulin) to treat or manage diabetes, it is desirable to have systems, devices, or methods that can automatically utilize glucose information collected by an analyte monitoring system to provide medication dose guidance in a readily accessible manner on an as-needed basis. It is further desirable for such systems, devices, or methods to take into account the physiology, diet, activity, and/or behavior of a user or patient to be treated in providing such medication dose guidance, as such may improve accuracy and reliability. Further, in some circumstances, it is also desirable for such systems, devices, or methods to be capable of automatically delivering a selected medication dose.

Current ADA (American Diabetes Association) standards of care are vague when describing when and how basal insulin should be titrated to improve glucose control, prescribing that care providers use an “evidence-based titration method.” Clinically evaluated basal insulin titration methods are typically based on readings of discrete blood glucose finger sticks taken while fasting. Medication decision making based on such sparse data can be problematic, especially for a drugs like long-acting basal insulin, where physiological effects may be conferred for up to 42 hours after administration. Indeed, acute responses like nocturnal hypoglycemia may not be recorded if the titration method relies only upon a morning fasting blood glucose reading. Moreover, blood glucose draws present a burden to the patient to gather.

For these and other reasons, needs exist for improved systems, methods, and devices relating to medication dose guidance.

### SUMMARY

Provided herein are example embodiments of systems, devices and methods relating to the provision of medication dose guidance and, in some embodiments, medication delivery. According to one aspect, many of the embodiments described herein comprise a dose guidance system (DGS) that includes a display device, a sensor control device, and a medication delivery device. The dose guidance system can include a dose guidance application (e.g., software) that can determine and output dose guidance (e.g., recommendations regarding dose amounts, corrections, and titrations) to a patient. Furthermore, according to some embodiments, the dose guidance system can learn a patient's dosing strategy during a learning period in which the dose guidance system can estimate key dosing parameters. According to some embodiments, the dose guidance system can also provide guidance for titrations and corrections once the system is configured with a patient's current dosing strategies. The dose guidance system can also provide guidance for basal insulin doses and adjustments. Exemplary system and safety features of the dose guidance system are also described.

In one embodiment, this system includes an auto-titration application that can provide dose change recommendations directly to the patient or to an HCP. In some embodiments, the auto-titration application may have inputs that provide some level of control for the HCP, such as a maximum dose amount limit that can be recommended, an adjustment of the amount of insulin that can be changed at one time, and/or a specification of the type of insulin. In addition, the auto-titration application may have additional outputs for the HCP, such as that the maximum recommended dose has been reached, the titration optimization has been reached and the patient is in good glucose control, and/or titration optimization has been reached and the patient remains in poor glucose control indicating that therapy escalation may be required. These outputs may be directed to the HCP in many ways: a) sent electronically, b) displayed in the app to the patient, with guidance to inform their HCP, and/or c) provide in reports that the HCP may access at their convenience.

Many of the embodiments provided herein comprise improved software features or graphical user interfaces for use with analyte monitoring systems that are highly intuitive, user-friendly, and provide for rapid access to physiological information of a user. More specifically, these embodiments allow a user (or an HCP) to rapidly determine an appropriate medication therapy based on information relating to the user's physiological conditions, historic dosing patterns, and other factors, without requiring the user (or an HCP) to go through the arduous task of examining large volumes of analyte data. Furthermore, some of the GUIs and GUI features, allow for users (and their caregivers) to better understand and improve a user's dosing patterns and subsequent hypo and hyperglycemic episodes. Likewise, many other embodiments provided herein comprise improved software features for dose guidance systems that improve upon: dose guidance provided to users by allowing for safe titration strategies that minimize hypoglycemic episodes and consideration of real-world occurrences that effect dosing strategies, to name only a few.

The systems described herein include a CGM-based algorithm for basal insulin titration that may improve upon current methods by analyzing the totality of a user's glucose for improved dose optimization. Possible clinical implementations for the algorithm are also presented. Given the effect that clinical inertia and delayed therapy intensification has on poor outcomes in Type 2 diabetes, a tool to aid patients and their care providers in dose titration represents an important step forward for CGM-based diabetes management.

Systems, devices and methods are provided for titration of a medication dose for a patient or user. The titration may be based on determining a hypoglycemic risk of a user for a plurality of time-of-day periods. The determination of the hypoglycemic risk may be accomplished by known methods, including the glucose pattern analysis, glucose dysregulation analysis, low alarm frequency analysis, and combinations thereof. The system may then select a recommended action based on the analyte pattern type. The system may then store an indicator of the recommended action in a computer memory for output. The system may then output the recommended action. The recommended action may be outputted to the user, an HCP, or a caregiver. The recommended action may also vary depending on who is receiving the recommended action.

Other systems, devices, methods, features and advantages of the subject matter described herein will be or will become apparent to one with skill in the art upon examination of the following figures and detailed description. It is intended that all such additional systems, devices, methods, features and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIGS. 1A and 1B are block diagrams of example embodiments of a dose guidance system.

FIG. 2A is a schematic diagram depicting an example embodiment of a sensor control device.

FIG. 2B is a block diagram depicting an example embodiment of a sensor control device.

FIG. 3A is a schematic diagram depicting an example embodiment of a medication delivery device.

FIG. 3B is a block diagram depicting an example embodiment of a medication delivery device.

FIG. 4A is a schematic diagram depicting an example embodiment of a display device.

FIG. 4B is a block diagram depicting an example embodiment of a display device.

FIG. 5 is a block diagram depicting an example embodiment of a user interface device.

FIG. 6A is a flow diagram depicting an example embodiment of a process flow for operations by a dose guidance application for assessing a meal bolus titration for a multiple daily injection (MDI) dosing therapy.

FIG. 6B is a flow diagram depicting an example embodiment of a process flow for operations by a dose guidance application for a glucose pattern analysis (GPA).

FIG. 6C is an example embodiment of a graph depicting information for determining a hypoglycemia risk and other metrics for a GPA.

FIGS. 7A-7B are flow diagrams depicting example embodiments of a process flow for operations by a dose guidance application for a glucose dysregulation analysis.

FIGS. 8A-8B are flow diagrams depicting example embodiments of a process flow for operations by a dose guidance application for a low alarm frequency analysis.

FIG. 9 is a flow diagram depicting an example embodiment of a process flow for operations by a dose guidance application based on multiple analytical methods.

FIG. 10 is a flow diagram depicting an example embodiment of a process flow for operations by a dose guidance application for determining optimal control.

### DETAILED DESCRIPTION

Before the present subject matter is described in detail, it is to be understood that this disclosure is not limited to the particular embodiments described herein, as such may, of course, vary. It is also to be understood that the terminology used herein is for the purpose of describing particular embodiments only, and is not intended to be limiting, since the scope of the present disclosure will be limited only by the appended claims.

Generally, embodiments of the present disclosure include systems, devices, and methods related to medication dose guidance. The dose guidance can be based on a broad array of information and categories of information specific to a user, such as the user's current and prior analyte levels, the user's current and prior diet, the user's current and prior physical activities, the user's current and prior medication history (including dosing logs), and other physiological information about the user. According to one aspect of the embodiments, the dose guidance provided by the systems, devices, and methods of the present disclosure can be based—not only on individual categories of information—but also on the predicted impact of such categories of information on the user's future analyte levels.

The dose guidance functionality can be implemented as a dose guidance application (DGA) that includes software and/or firmware instructions stored in a memory of a computing device for execution by at least one processor or processing circuitry thereof. The computing device can be in the possession of a user or healthcare professional (HCP), and the user or HCP can interface with the computing device through a user interface. According to some embodiments, the computing device can be a server or trusted computer system that is accessible through a network, and the dose guidance software can be presented to the user in the form of an interactive web page by way of a browser executed on a local display device (having the user interface) in communication with the server or trusted computer system through the network. In this and other embodiments, the dose guidance software can be executed across multiple devices, or executed, in part, on processing circuitry of a local display device and, in part, on processing circuitry of a server or trusted computer system. It will be understood by those of skill in the art that when the DGA is described as performing an action, such action is performed according to instructions stored in a computer memory (including instructions hardcoded in read only memory) that, when executed by at least one processor of at least one computing device, causes the DGA to perform the described action. In all cases the action can alternatively be performed by hardware that is hardwired to implement the action (e.g., dedicated circuitry) as opposed to performance by way of instructions stored in memory.

Furthermore, as used herein, a system on which the DGA is implemented can be referred to as a dose guidance system. The dose guidance system can be configured for the sole purpose of providing dose guidance or can be a multifunctional system of which dose guidance is only one aspect. For example, in some embodiments the dose guidance system can also be capable of monitoring analyte levels of a user. In some embodiments the dose guidance system can also be capable of delivering medication to the user, such as with an injection or infusion device. In some embodiments, the dose guidance system is capable of both monitoring analytes and delivering medication.

These embodiments and others described herein represent improvements in the field of computer-based dose determination, analyte monitoring, and medication delivery systems. The specific features and potential advantages of the disclosed embodiments are further discussed below.

Before describing the dose guidance embodiments in detail, it is first desirable to describe examples of dose guidance systems on or through which the dose guidance application can be implemented.

### Example Embodiments of Dose Guidance Systems

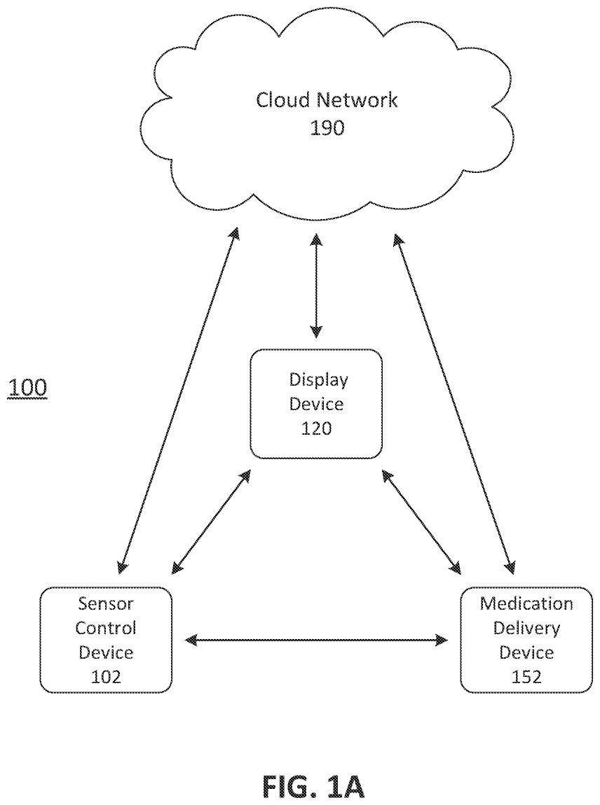

FIG. 1A is a block diagram depicting an example embodiment of dose guidance system 100. In this embodiment, dose guidance system 100 is capable of providing dose guidance, monitoring one or more analytes, and delivering one or more medications. This multifunctional example is used to illustrate the high degree of interconnectivity and performance obtainable by system 100. However, in the embodiments described herein, the analyte monitoring components, the medication delivery components, or both can be omitted if desired.

Here, system 100 includes a sensor control device (SCD) 102 configured to collect analyte level information from a user, a medication delivery device (MDD) 152 configured to deliver medication to the user, and a display device 120 configured to present information to the user and receive input or information from the user. The structure and function of each device will be described in detail herein.

System 100 is configured for highly interconnected and highly flexible communication between devices. Each of the three devices 102, 120, and 152, can communicate directly with each other (without passing through an intermediate electronic device) or indirectly with each other (such as through cloud network 190, or through another device and then through network 190). Bidirectional communication capability between devices, as well as between devices and network 190, is shown in FIG. 1A with a double-sided arrow. However, those of skill in the art will appreciate that any of the one or more devices (e.g., SCD) can be capable of unidirectional communication such as, for example, broadcasting, multicasting, or advertising communications. In each instance, whether bidirectional or unidirectional, the communication can be wired or wireless. The protocols that govern communication over each path can be the same or different, and can be either proprietary or standardized. For example, wireless communication between devices 102, 120, and 152 can be performed according to a Bluetooth (including Bluetooth Low Energy) standard, a Near Field Communication (NFC) standard, a Wi-Fi (802.11x) standard, a mobile telephony standard, or others. All communications over the various paths can be encrypted, and each device of FIG. 1A can be configured to encrypt and decrypt those communications sent and received. In each instance the communication pathways of FIG. 1A can be direct (e.g., Bluetooth or NFC) or indirect (e.g., Wi-Fi, mobile telephony, or other internet protocol). Embodiments of system 100 do not need to have the capability to communicate across all of the pathways indicated in FIG. 1A.

In addition, although FIG. 1A depicts a single display device 120, a single SCD 102, and a single MDD 152, those of skill in the art will appreciate that system 100 can comprise a plurality of any of the aforementioned devices. By way of example only, system 100 can comprise a single SCD 102 in communication with multiple (e.g., two, three, four, etc.) display devices 120 and/or multiple MDDs 152. Alternatively, system 100 can comprise a plurality of SCDs 102 in communication with a single display device 120 and/or a single MDD 152. Furthermore, each of the plurality of devices can be of the same or different device types. For example, system 100 can comprise multiple display devices 120, including a smart phone, a handheld receiver, and/or a smart watch, each of which can be in communication with SCD 102 and/or MDD 152, as well in communication with each other.

Analyte data can be transferred between each device within system 100 in an autonomous fashion (e.g., transmitting automatically according to a schedule), or in response to a request for analyte data (e.g., sending a request from a first device to a second device for analyte data, followed by transmission of the analyte data from the second device to the first device). Other techniques for communicating data can also be employed to accommodate more complex systems like cloud network 190.

FIG. 1B is a block diagram depicting another example embodiment of dose guidance system 100. Here, system 100 includes SCD 102, MDD 152, a first display device 120-1, a second display device 120-2, local computer system 170, and trusted computer system 180 that is accessible by cloud network 190. SCD 102 and MDD 152 are capable of communication with each other and with display device 120-1, which can act as a communication hub for aggregating information from SCD 102 and MDD 152, processing and displaying that information where desired, and transferring some or all of the information to cloud network 190 and/or computer system 170. Conversely, display device 120-1 can receive information from cloud network 190 and/or computer system 170 and communicate some or all of the received information to SCD 102, MDD 152, or both. Computer system 170 may be a personal computer, a server terminal, a laptop computer, a tablet, or other suitable data processing device. Computer system 170 can include or present software for data management and analysis and communication with the components in system 100. Computer system 170 can be used by the user or a medical professional to display and/or analyze analyte data measured by SCD 102. Furthermore, although FIG. 1B depicts a single SCD 102, a single MDD 152, and two display devices 120-1 and 120-2, those of skill in the art will appreciate that system 100 can include a plurality of any of the aforementioned devices, wherein each plurality of devices can comprise the same or different types of devices.

Referring still to FIG. 1B, according to some embodiments, trusted computer system 180 can be within the possession of a manufacturer or distributor of a component of system 100, either physically or virtually through a secured connection, and can be used to perform authentication of the devices of system 100 (e.g., devices 102, 120-n, 152), for secure storage of the user's data, and/or as a server that serves a data analytics program (e.g., accessible via a web browser) for performing analysis on the user's measured analyte data and medication history. Trusted computer system 180 can also act as a data hub for routing and exchanging data between all devices in communication with system 180 through cloud network 190. In other words, all devices of system 100 that are capable of communicating with cloud network 190 (e.g., either directly with an internet connection or indirectly via another device), are also capable of communicating with all of the other devices of system 100 that are capable of communicating with cloud network 190, either directly or indirectly.

Display device 120-2 is depicted in communication with cloud network 190. In this example, device 120-2 can be in the possession of another user that is granted access to the analyte and medication data of the person wearing SCD 102. For example, the person in possession of display device 120-2 can be a parent of a child wearing SCD 102, as one example, or a caregiver of an elderly patient wearing SCD 102, as another example. System 100 can be configured to communicate analyte and medication data about the wearer through cloud network 190 (e.g., via trusted computer system 180) to another user with granted access to the data.

### Example Embodiments of Analyte Monitoring Devices

The analyte monitoring functionality of dose guidance system 100 can be realized through inclusion of one or more devices capable of collecting, processing, and displaying analyte data of the user. Example embodiments of such devices and their methods of use are described in Int'l Publ. No. WO 2018/152241 and U.S. Patent Publ. No. 2011/0213225, both of which are incorporated by reference herein in their entireties for all purposes.

Analyte monitoring can be performed in numerous different ways. “Continuous Analyte Monitoring” devices (e.g., “Continuous Glucose Monitoring” devices), for example, can transmit data from a sensor control device to a display device continuously or repeatedly with or without prompting, e.g., automatically according to a schedule. “Flash Analyte Monitoring” devices (e.g., “Flash Glucose Monitoring” devices or simply “Flash” devices), as another example, can transfer data from a sensor control device in response to a user-initiated request for data by a display device (e.g., a scan), such as with a Near Field Communication (NFC) or Radio Frequency Identification (RFID) protocol.

Analyte monitoring devices that utilize a sensor configured to be placed partially or wholly within a user's body can be referred to as in vivo analyte monitoring devices. For example, an in vivo sensor can be placed in the user's body such that at least a portion of the sensor is in contact with a bodily fluid (e.g., interstitial (ISF) fluid such as dermal fluid in the dermal layer or subcutaneous fluid beneath the dermal layer, blood, or others) and can measure an analyte concentration in that bodily fluid. In vivo sensors can use various types of sensing techniques (e.g., chemical, electrochemical, or optical). Some systems utilizing in vivo analyte sensors can also operate without the need for finger stick calibration.

“In vitro” devices are those where a sensor is brought into contact with a biological sample outside of the body (or rather “ex vivo”). These devices typically include a port for receiving an analyte test strip carrying bodily fluid of the user, which can be analyzed to determine the user's blood glucose level. Other ex vivo devices have been proposed that attempt to measure the user's internal analyte level non-invasively, such as by using an optical technique that can measure an internal body analyte level without mechanically penetrating the user's body or skin. In vivo and ex vivo devices often include in vitro capability (e.g., an in vivo display device that also includes a test strip port).

The present subject matter will be described with respect to sensors capable of measuring a glucose concentration, although detection and measurement of concentrations of other analytes are within the scope of the present disclosure. These other analytes can include, for example, ketones, lactate, oxygen, hemoglobin A1C, acetyl choline, amylase, bilirubin, cholesterol, chorionic gonadotropin, creatine kinase (e.g., CK-MB), creatine, DNA, fructosamine, glutamine, growth hormones, hormones, peroxide, prostate-specific antigen, prothrombin, RNA, thyroid stimulating hormone, troponin and others. The concentration of drugs, such as, for example, antibiotics (e.g., gentamicin, vancomycin, and the like), digitoxin, digoxin, drugs of abuse, theophylline, and warfarin, may also be monitored. The sensor can be configured to measure two or more different analytes at the same or different times. In some embodiments, the sensor control device can be coupled with two or more sensors, where one sensor is configured to measure a first analyte (e.g., glucose) and the other one or more sensors are configured to measure one or more different analytes (e.g., any of those described herein). In other embodiments, a user can wear two or more sensor control devices, each of which is capable of measuring a different analyte.

The embodiments described herein can be used with all types of in vivo, in vitro, and ex vivo devices capable of monitoring the aforementioned analytes and others.

In many embodiments, the sensor operation can be controlled by SCD 102. The sensor can be mechanically and communicatively coupled with SCD 102, or can be just communicatively coupled with SCD 102 using a wireless communication technique. SCD 102 can include the electronics and power supply that enable and control analyte sensing performed by the sensor. In some embodiments the sensor or SCD 102 can be self-powered such that a battery is not required. SCD 102 can also include communication circuitry for communicating with another device that may or may not be local to the user's body (e.g., a display device). SCD 102 can reside on the body of the user (e.g., attached to or otherwise placed on the user's skin, or carried in the user's clothes, etc.). SCD 102 can also be implanted within the body of the user along with the sensor. Functionality of SCD 102 can be divided between a first component implanted within the body (e.g., a component that controls the sensor) and a second component that resides on or otherwise outside the body (e.g., a relay component that communicates with the first component and also with an external device like a computer or smartphone). In other embodiments, SCD 102 can be external to the body and configured to non-invasively measure the user's analyte levels. The sensor control device, depending on the actual implementation or embodiment, can also be referred to as a “sensor control unit,” an “on-body electronics” device or unit, an “on-body” device or unit, an “in body electronics” device or unit, an “in-body” device or unit, or a “sensor data communication” device or unit, to name a few.

In some embodiments, SCD 102 may include a user interface (e.g., a touchscreen) and be capable of processing the analyte data and displaying the resultant calculated analyte levels to the user. In such cases, the dose guidance embodiments described herein can be implemented directly by SCD 102, in whole or in part. In many embodiments, the physical form factor of SCD 102 is minimized (e.g., to minimize the appearance on the user's body) or the sensor control device may be inaccessible to the user (e.g., if wholly implanted), or other factors may make it desirable to have a display device usable by the user to read analyte levels and interface with the sensor control device.

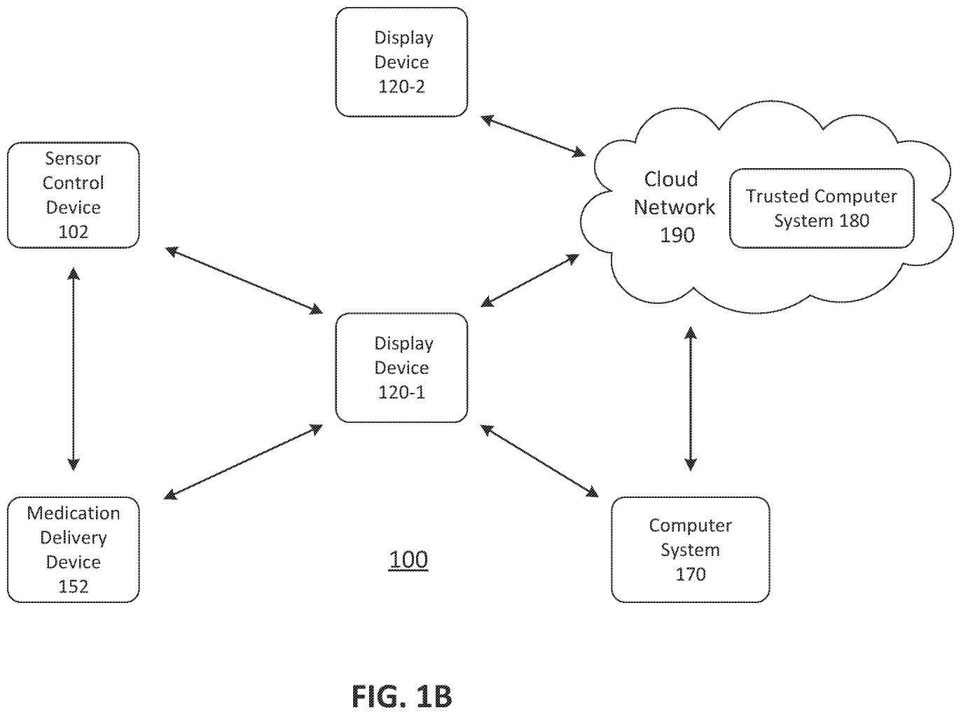

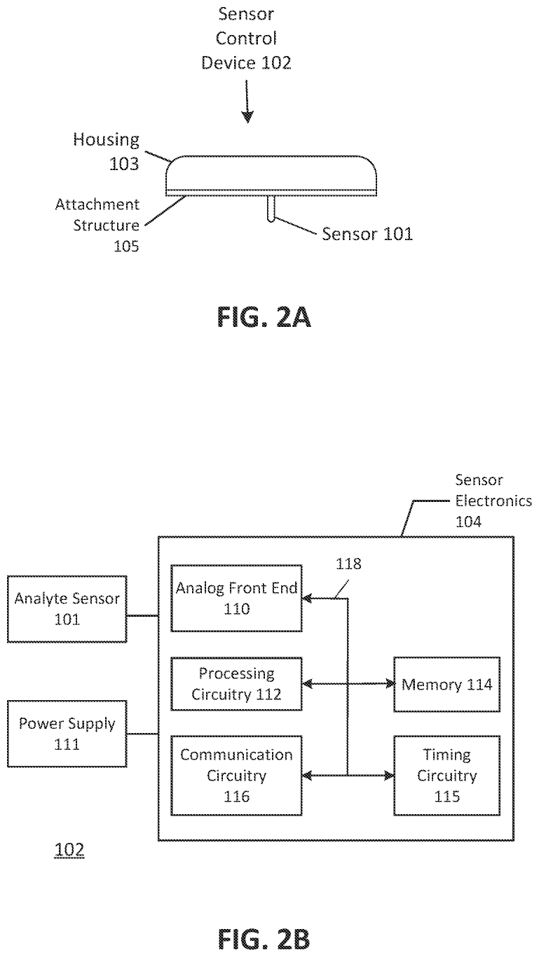

FIG. 2A is a side view of an example embodiment of SCD 102. SCD 102 can include a housing or mount 103 for sensor electronics (FIG. 2B), which can be electrically coupled with an analyte sensor 101, which is configured here as an electrochemical sensor. According to some embodiments, sensor 101 can be configured to reside partially within a user's body (e.g., through an exterior-most surface of the skin) where it can make fluid contact with a user's bodily fluid and be used, along with the sensor electronics, to measure analyte-related data of the user. A structure for attachment 105, such as an adhesive patch, can be used to secure housing 103 to a user's skin. Sensor 101 can extend through attachment structure 105 and project away from housing 103. Those of skill in the art will appreciate that other forms of attachment to the body and/or housing 103 may be used, in addition to or instead of adhesive, and are fully within the scope of the present disclosure.

SCD 102 can be applied to the body in any desired manner. For example, an insertion device (not shown), sometimes referred to as an applicator, can be used to position all or a portion of analyte sensor 101 through an external surface of the user's skin and into contact with the user's bodily fluid. In doing so, the insertion device can also position SCD 102 onto the skin. In other embodiments, the insertion device can position sensor 101 first, and then accompanying electronics (e.g., wireless transmission circuitry and/or data processing circuitry, and the like) can be coupled with sensor 101 afterwards (e.g., inserted into a mount), either manually or with the aid of a mechanical device. Examples of insertion devices are described in U.S. Patent Publ. Nos. 2008/0009692, 2011/0319729, 2015/0018639, 2015/0025345, and 2015/0173661, 2018/0235520, all which are incorporated by reference herein in their entireties for all purposes.

FIG. 2B is a block diagram depicting an example embodiment of SCD 102 having analyte sensor 101 and sensor electronics 104. Sensor electronics 104 can be implemented in one or more semiconductor chips (e.g., an application specific integrated circuit (ASIC), processor or controller, memory, programmable gate array, and others). In the embodiment of FIG. 1B, sensor electronics 104 includes high-level functional units, including an analog front end (AFE) 110 configured to interface in an analog manner with sensor 101 and convert analog signals to and/or from digital form (e.g., with an A/D converter), a power supply 111 configured to supply power to the components of SCD 102, processing circuitry 112, memory 114, timing circuitry 115 (e.g., such as an oscillator and phase locked loop for providing a clock or other timing to components of SCD 102), and communication circuitry 116 configured to communicate in wired and/or wireless fashion with one or more devices external to SCD 102, such as display device 120 and/or MDD 152.

SCD 102 can be implemented in a highly interconnected fashion, where power supply 111 is coupled with each component shown in FIG. 2B and where those components that communicate or receive data, information, or commands (e.g., AFE 110, processing circuitry 112, memory 114, timing circuitry 115, and communication circuitry 116), can be communicatively coupled with every other such component over, for example, one or more communication connections or buses 118.

Processing circuitry 112 can include one or more processors, microprocessors, controllers, and/or microcontrollers, each of which can be a discrete chip or distributed amongst (and a portion of) a number of different chips. Processing circuitry 112 can include on-board memory. Processing circuitry 112 can interface with communication circuitry 116 and perform analog-to-digital conversions, encoding and decoding, digital signal processing and other functions that facilitate the conversion of data signals into a format (e.g., in-phase and quadrature) suitable for wireless or wired transmission. Processing circuitry 112 can also interface with communication circuitry 116 to perform the reverse functions necessary to receive a wireless transmission and convert it into digital data or information.

Processing circuitry 112 can execute instructions stored in memory 114. These instructions can cause processing circuitry 112 to process raw analyte data (or pre-processed analyte data) and arrive at a final calculated analyte level. In some embodiments, instructions stored in memory 114, when executed, can cause processing circuitry 112 to process raw analyte data to determine one or more of: a calculated analyte level, an average calculated analyte level within a predetermined time window, a calculated rate-of-change of an analyte level within a predetermined time window, and/or whether a calculated analyte metric exceeds a predetermined threshold condition. These instructions can also cause processing circuitry 112 to read and act on received transmissions, to adjust the timing of timing circuitry 115, to process data or information received from other devices (e.g., calibration information, encryption or authentication information received from display device 120, and others), to perform tasks to establish and maintain communication with display device 120, to interpret voice commands from a user, to cause communication circuitry 116 to transmit, and others. In embodiments where SCD 102 includes a user interface, then the instructions can cause processing circuitry 112 to control the user interface, read user input from the user interface, cause the display of information on the user interface, format data for display, and others. The functions described here that are coded in the instructions can instead be implemented by SCD 102 with the use of a hardware or firmware design that does not rely on the execution of stored software instructions to accomplish the functions.

Memory 114 can be shared by one or more of the various functional units present within SCD 102, or can be distributed amongst two or more of them (e.g., as separate memories present within different chips). Memory 114 can also be a separate chip of its own. Memory 114 is non-transitory, and can be volatile (e.g., RAM, etc.) and/or non-volatile memory (e.g., ROM, flash memory, F-RAM, etc.).

Communication circuitry 116 can be implemented as one or more components (e.g., transmitter, receiver, transceiver, passive circuit, encoder, decoder, and/or other communication circuitry) that perform the functions for communications over the respective communications paths or links. Communication circuitry 116 can include or be coupled to one or more antenna for wireless communication.

Power supply 111 can include one or more batteries, which can be rechargeable or single-use disposable batteries. Power management circuitry can also be included to regulate battery charging and monitor usage of power supply 111, boost power, perform DC conversions, and the like.

Additionally, an on-skin or sensor temperature reading or measurement can be collected by an optional temperature sensor (not shown). Those readings or measurements can be communicated (either individually or as an aggregated measurement over time) from SCD 102 to another device (e.g., display device 120). The temperature reading or measurement, however, can be used in conjunction with a software routine executed by SCD 102 or display device 120 to correct or compensate the analyte measurement output to the user, instead of or in addition to, actually outputting the temperature measurement to the user.

### Example Embodiments of Medication Delivery Devices

The medication delivery functionality of dose guidance system 100 can be realized through inclusion of one or more medication delivery devices (MDDs) 152. MDD 152 can be any device configured to deliver a specific dose of medication. The MDD 152 can also include devices that transmit data regarding doses to the DGA, e.g., pen caps, even though the device itself may not deliver the medication. The MDD 152 can be configured as a portable injection device (PID) that can deliver a single dose per one injection, such as a bolus. The PID can be a basic manually-operated syringe, where the medication is either preloaded in the syringe or must be drawn into the syringe from a container prior to injection. In most embodiments, however, the PID includes electronics for interfacing with the user and performing the delivery of the medication. PIDs are often referred to as medication pens, although a pen-like appearance is not required. PIDs having user interface electronics are often referred to as smart pens. PIDs can be used to deliver one dose and then disposed of, or can be durable and used repeatedly to deliver many doses over the course of a day, week, or month. PIDs are often relied upon by users that practice a multiple daily injection (MDI) therapy regimen.

The MDD can also comprise a pump and infusion set. The infusion set includes a tubular cannula that resides at least partially within the recipient's body. The tubular cannula is in fluid communication with a pump, which can deliver medication through the cannula and into the recipient's body in small increments repeatedly over time. The infusion set can be applied to the recipient's body using an infusion set applicator, and the infusion set often stays implanted for 2 to 3 days or longer. A pump device includes electronics for interfacing with the user and for controlling the slow infusion of the medication. Both a PID and a pump can store the medication in a medication reservoir.

MDD 152 can function as part of a closed-loop system (e.g., an artificial pancreas system requiring no user intervention to operate), semi-closed loop system (e.g., an insulin loop system requiring seldom user intervention to operate, such as to confirm changes in dose), or an open loop system. For example, the diabetic's analyte level can be monitored in a repeated automatic fashion by SCD 102, and that information can be used by the dose guidance embodiments described herein to automatically calculate or otherwise determine the appropriate drug dosage to control the diabetic's analyte level and subsequently deliver that dose to the diabetic's body. This calculation can occur within MDD 152 or any other device of system 100 and the resulting determined dosage can then be communicated to MCD 152.

In many embodiments, the dose guidance provided by the embodiments described herein will be for a type of insulin (e.g., rapid-acting (RA), short-acting insulin, intermediate-acting insulin (e.g., NPH insulin), long-acting (LA), ultra long-acting insulin, and mixed insulin), and will be the same medication delivered by MDD 152. The type of insulin includes human insulin and synthetic insulin analogs. The insulin can also include premixed formulations. However, the dose guidance embodiments set forth herein and the medication delivery capabilities of MDD 152 can be applied to other non-insulin medications. Such medications can include, but are not limited to exenatide, exenatide extended release, liraglutide, lixisenatide, semaglutide, pramlintide, metformin, SLGT1-i inhibitors, SLGT2-i inhibitors, and DPP4 inhibitors. The dose guidance embodiments can also include combination therapies. Combination therapies can include, but are not limited to, insulin and glucagon-like peptide-1 receptor agonists (GLP-1 RA), insulin and pramlintide.

For ease of description of the dose guidance embodiments herein, MDD 152 will often be described in the form of a PID, specifically a smart pen. However, those of skill in the art will readily understand that MDD 152 can alternatively be configured as a pen cap, a pump, or any other type of medication delivery device.

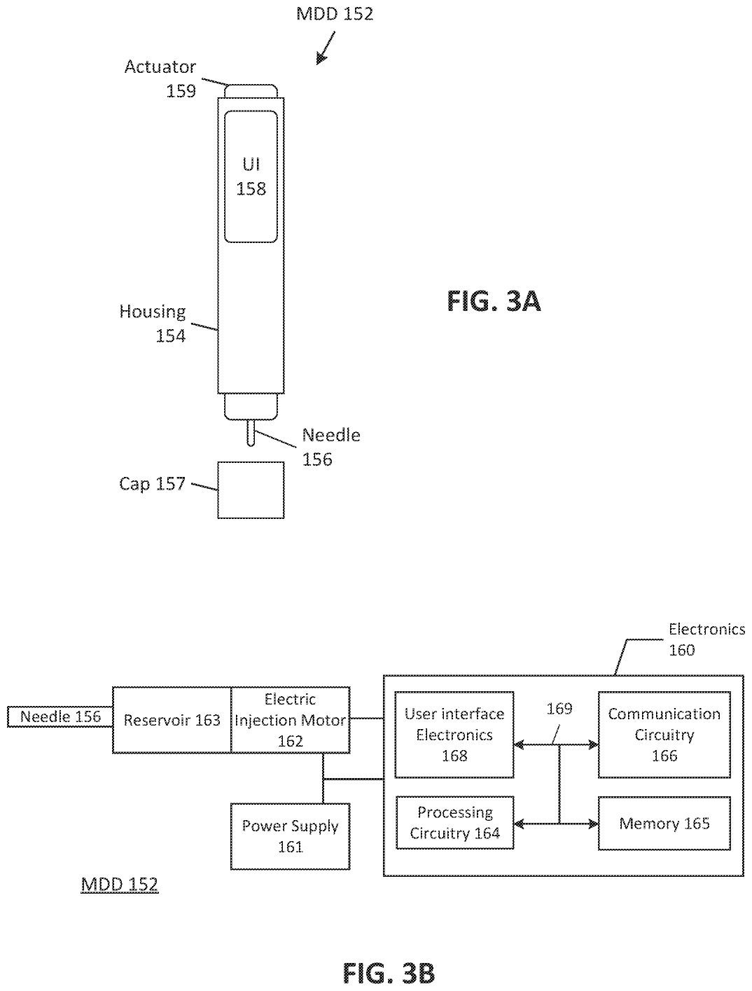

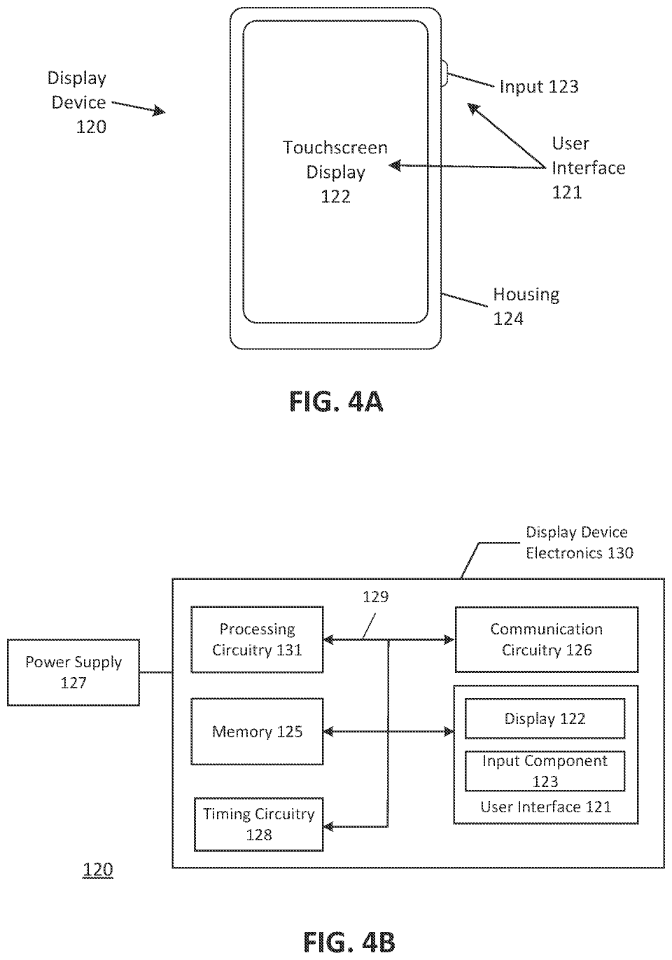

FIG. 3A is schematic diagram depicting an example embodiment of an MDD 152 configured as a PID, specifically a smart pen. MDD 152 can include a housing 154 for electronics, an injection motor, and a medication reservoir (see FIG. 3B), from which medication can be delivered through needle 156. Housing 154 can include a removable or detachable cap or cover 157 that, when attached, can shield needle 156 when not in use, and then be detached for injection. MDD 152 can also include a user interface 158 which can be implemented as a single component (e.g., a touchscreen for outputting information to the user and receiving input from the user) or as multiple components (e.g., a touchscreen or display in combination with one or more buttons, switches, or the like). MDD 152 can also include an actuator 159 that can be moved, depressed, touched or otherwise activated to initiate delivery of the medication from an internal reservoir through needle 156 and into the recipient's body. According to some embodiments, cap 157 and actuator 159 can also include one or more safety mechanisms to prevent removal and/or actuation to mitigate risk of a harmful medication injection. Details of these safety mechanisms and others are described in U.S. Patent Publ. No. 2019/0343385 (the '385 publication), which is hereby incorporated in its entirety for all purposes.

FIG. 3B is a block diagram depicting an example embodiment of MDD 152 having electronics 160, coupled with a power supply 161 and an electric injection motor 162, which in turn is coupled with power supply 161 and a medication reservoir 163. Needle 156 is shown in fluid communication with reservoir 163, and a valve (not shown) may be present between reservoir 163 and needle 156. Reservoir 163 can be permanent or can be removable and replaced with another reservoir containing the same or different medication. Electronics 160 can be implemented in one or more semiconductor chips (e.g., an application specific integrated circuit (ASIC), processor or controller, memory, programmable gate array, and others). In the embodiment of FIG. 3B, electronics 160 can include high-level functional units, including processing circuitry 164, memory 165, communication circuitry 166 configured to communicate in wired and/or wireless fashion with one or more devices external to MDD 152 (such as display device 120), and user interface electronics 168.

MDD 152 can be implemented in a highly interconnected fashion, where power supply 161 is coupled with each component shown in FIG. 3B and where those components that communicate or receive data, information, or commands (e.g., processing circuitry 164, memory 165, and communication circuitry 166), can be communicatively coupled with every other such component over, for example, one or more communication connections or buses 169.

Processing circuitry 164 can include one or more processors, microprocessors, controllers, and/or microcontrollers, each of which can be a discrete chip or distributed amongst (and a portion of) a number of different chips. Processing circuitry 164 can include on-board memory. Processing circuitry 164 can interface with communication circuitry 166 and perform analog-to-digital conversions, encoding and decoding, digital signal processing and other functions that facilitate the conversion of data signals into a format (e.g., in-phase and quadrature) suitable for wireless or wired transmission. Processing circuitry 164 can also interface with communication circuitry 166 to perform the reverse functions necessary to receive a wireless transmission and convert it into digital data or information.

Processing circuitry 164 can execute software instructions stored in memory 165. These instructions can cause processing circuitry 164 to receive a selection or provision of a specified dose from a user (e.g., entered via user interface 158 or received from another device), process a command to deliver a specified dose (such as a signal from actuator 159), and control motor 162 to cause delivery of the specified dose. These instructions can also cause processing circuitry 164 to read and act on received transmissions, to process data or information received from other devices (e.g., calibration information, encryption or authentication information received from display device 120, and others), to perform tasks to establish and maintain communication with display device 120, to interpret voice commands from a user, to cause communication circuitry 166 to transmit, and others. In embodiments where MDD 152 includes user interface 158, then the instructions can cause processing circuitry 164 to control the user interface, read user input from the user interface (e.g., entry of a medication dose for administration or entry of confirmation of a recommended medication dose), cause the display of information on the user interface, format data for display, and others. The functions described here that are coded in the instructions can instead be implemented by MDD 152 with the use of a hardware or firmware design that does not rely on the execution of stored software instructions to accomplish the functions.

Memory 165 can be shared by one or more of the various functional units present within MDD 152, or can be distributed amongst two or more of them (e.g., as separate memories present within different chips). Memory 165 can also be a separate chip of its own. Memory 165 is non-transitory, and can be volatile (e.g., RAM, etc.) and/or non-volatile memory (e.g., ROM, flash memory, F-RAM, etc.).

Communication circuitry 166 can be implemented as one or more components (e.g., transmitter, receiver, transceiver, passive circuit, encoder, decoder, and/or other communication circuitry) that perform the functions for communications over the respective communications paths or links. Communication circuitry 166 can include or be coupled to one or more antenna for wireless communication. Details of exemplary antenna can be found in the '385 publication, which is hereby incorporated in its entirety for all purposes.

Power supply 161 can include one or more batteries, which can be rechargeable or single-use disposable batteries. Power management circuitry can also be included to regulate battery charging and monitor usage of power supply 161, boost power, perform DC conversions, and the like.

MDD 152 may also include an integrated or attachable in vitro glucose meter, including an in vitro test strip port (not shown) to receive an in vitro glucose test strip for performing in vitro blood glucose measurements.

### Example Embodiments of Display Devices

Display device 120 can be configured to display information pertaining to system 100 to the user and accept or receive input from the user also pertaining to system 100. Display device 120 can display recent measured analyte levels, in any number of forms, to the user. The display device can display historical analyte levels of the user as well as other metrics that describe the user's analyte information (e.g., time in range, ambulatory glucose profile (AGP), hypoglycemia risk levels, etc.). Display device 120 can display medication delivery information, such as historical dose information and the times and dates of administration. Display device 120 can display alarms, alerts, or other notifications pertaining to analyte levels and/or medication delivery.

Display device 120 can be dedicated for use with system 100 (e.g., an electronic device designed and manufactured for the primary purpose of interfacing with an analyte sensor and/or a medication delivery device), as well as devices that are multifunctional, general purpose computing devices such as a handheld or portable mobile communication device (e.g., a smartphone or tablet), or a laptop, personal computer, or other computing device. Display device 120 can be configured as a mobile smart wearable electronics assembly, such as a smart glass or smart glasses, or a smart watch or wristband. Display devices, and variations thereof, can be referred to as “reader devices,” “readers,” “handheld electronics” (or handhelds), “portable data processing” devices or units, “information receivers,” “receiver” devices or units (or simply receivers), “relay” devices or units, or “remote” devices or units, to name a few.

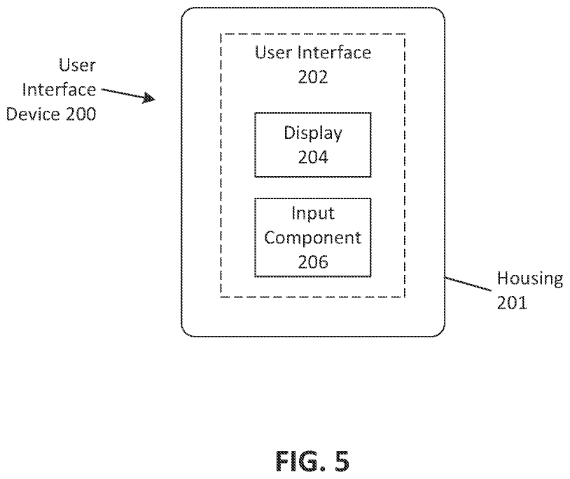

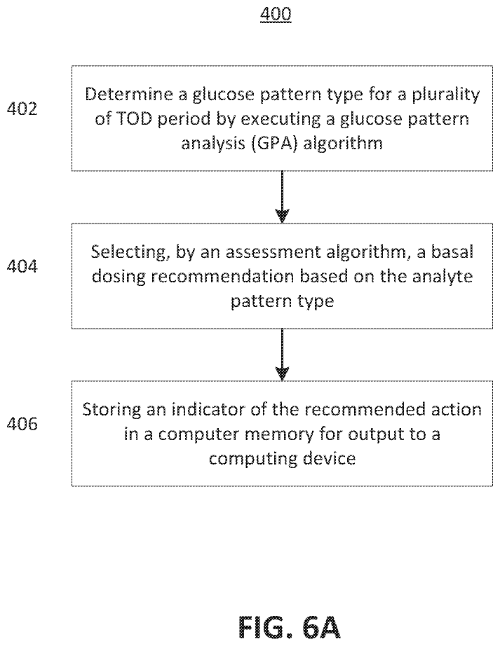

FIG. 4A is a schematic view depicting an example embodiment of display device 120. Here, display device 120 includes a user interface 121 and a housing 124 in which display device electronics 130 (FIG. 4B) are held. User interface 121 can be implemented as a single component (e.g., a touchscreen capable of input and output) or multiple components (e.g., a display and one or more devices configured to receive user input). In this embodiment, user interface 121 includes a touchscreen display 122 (configured to display information and graphics and accept user input by touch) and an input button 123, both of which are coupled with housing 124.

Display device 120 can have software stored thereon (e.g., by the manufacturer or downloaded by the user in the form of one or more “apps” or other software packages) that interface with SCD 102, MDD 152, and/or the user. In addition, or alternatively, the user interface can be affected by a web page displayed on a browser or other internet interfacing software executable on display device 120.

FIG. 4B is a block diagram of an example embodiment of a display device 120 with display device electronics 130. Here, display device 120 includes user interface 121 including display 122 and an input component 123 (e.g., a button, actuator, touch sensitive switch, capacitive switch, pressure sensitive switch, jog wheel, microphone, speaker, or the like), processing circuitry 131, memory 125, communication circuitry 126 configured to communicate to and/or from one or more other devices external to display device 120), a power supply 127, and timing circuitry 128 (e.g., such as an oscillator and phase locked loop for providing a clock or other timing to components of SCD 102). Each of the aforementioned components can be implemented as one or more different devices or can be combined into a multifunctional device (e.g., integration of processing circuitry 131, memory 125, and communication circuitry 126 on a single semiconductor chip). Display device 120 can be implemented in a highly interconnected fashion, where power supply 127 is coupled with each component shown in FIG. 4B and where those components that communicate or receive data, information, or commands (e.g., user interface 121, processing circuitry 131, memory 125, communication circuitry 126, and timing circuitry 128), can be communicatively coupled with every other such component over, for example, one or more communication connections or buses 129. FIG. 4B is an abbreviated representation of the typical hardware and functionality that resides within a display device and those of ordinary skill in the art will readily recognize that other hardware and functionality (e.g., codecs, drivers, glue logic) can also be included.

Processing circuitry 131 can include one or more processors, microprocessors, controllers, and/or microcontrollers, each of which can be a discrete chip or distributed amongst (and a portion of) a number of different chips. Processing circuitry 131 can include on-board memory. Processing circuitry 131 can interface with communication circuitry 126 and perform analog-to-digital conversions, encoding and decoding, digital signal processing and other functions that facilitate the conversion of data signals into a format (e.g., in-phase and quadrature) suitable for wireless or wired transmission. Processing circuitry 131 can also interface with communication circuitry 126 to perform the reverse functions necessary to receive a wireless transmission and convert it into digital data or information.

Processing circuitry 131 can execute software instructions stored in memory 125. These instructions can cause processing circuitry 131 to process raw analyte data (or pre-processed analyte data) and arrive at a corresponding analyte level suitable for display to the user. These instructions can cause processing circuitry 131 to read, process, and/or store a dose instruction from the user, and because the dose instruction to be communicated to MDD 152. These instructions can cause processing circuitry 131 to execute user interface software adapted to present an interactive group of graphical user interface screens to the user for the purposes of configuring system parameters (e.g., alarm thresholds, notification settings, display preferences, and the like), presenting current and historical analyte level information to the user, presenting current and historical medication delivery information to the user, collecting other non-analyte information from the user (e.g., information about meals consumed, activities performed, medication administered, and the like), and presenting notifications and alarms to the user. These instructions can also cause processing circuitry 131 to cause communication circuitry 126 to transmit, can cause processing circuitry 131 to read and act on received transmissions, to read input from user interface 121 (e.g., entry of a medication dose to be administered or confirmation of a recommended medication dose), to display data or information on user interface 121, to adjust the timing of timing circuitry 128, to process data or information received from other devices (e.g., analyte data, calibration information, encryption or authentication information received from SCD 102, and others), to perform tasks to establish and maintain communication with SCD 102, to interpret voice commands from a user, and others. The functions described here that are coded in the instructions can instead be implemented by display device 120 with the use of a hardware or firmware design that does not rely on the execution of stored software instructions to accomplish the functions.

Memory 125 can be shared by one or more of the various functional units present within display device 120, or can be distributed amongst two or more of them (e.g., as separate memories present within different chips). Memory 125 can also be a separate chip of its own. Memory 125 is non-transitory, and can be volatile (e.g., RAM, etc.) and/or non-volatile memory (e.g., ROM, flash memory, F-RAM, etc.).

Communication circuitry 126 can be implemented as one or more components (e.g., transmitter, receiver, transceiver, passive circuit, encoder, decoder, and/or other communication circuitry) that perform the functions for communications over the respective communications paths or links. Communication circuitry 126 can include or be coupled to one or more antenna for wireless communication.

Power supply 127 can include one or more batteries, which can be rechargeable or single-use disposable batteries. Power management circuitry can also be included to regulate battery charging and monitor usage of power supply 127, boost power, perform DC conversions, and the like.

Display device 120 can also include one or more data communication ports (not shown) for wired data communication with external devices such as computer system 170, SCD 102, or MDD 152. Display device 120 may also include an integrated or attachable in vitro glucose meter, including an in vitro test strip port (not shown) to receive an in vitro glucose test strip for performing in vitro blood glucose measurements.

Display device 120 can display the measured analyte data received from SCD 102 and can also be configured to output alarms, alert notifications, glucose values, etc., which may be visual, audible, tactile, or any combination thereof. In some embodiments, SCD 102 and/or MDD 152 can also be configured to output alarms, or alert notifications in visible, audible, tactile forms or combination thereof. Further details and other display embodiments can be found in, e.g., U.S. Patent Publ. No. 2011/0193704, which is incorporated herein by reference in its entirety for all purposes.

### Example Embodiments Related to Dose Guidance

The following example embodiments relate to dose guidance functionality provided by dose guidance system 100. The dose guidance functionality will, in many embodiments, be implemented as a set of software instructions stored and/or executed on one or more electronic devices. This dose guidance functionality will be referred to herein as a dose guidance application (DGA). In some embodiments, the DGA is stored, executed, and presented to the user on the same single electronic device. In other embodiments, the DGA can be stored and executed on one device, and presented to the user on a different electronic device. For example, the DGA can be stored and executed on trusted computer system 180 and presented to the user by way of a webpage displayed through an internet browser executed on display device 120. The DGA may be a stand-alone application or may be incorporated in whole or in part into another software application. The application may either be a mobile application, a web-based server that supports the mobile app by providing an alternate means of data processing and a communication hub or a combination of the two.

The system may either mobile-based, web-based, or a dual system that utilizes both entities. In a dual system, the algorithm may reside in either application. Within the mobile application, the user may receive daily reminders for basal dose administration and/or logging as an algorithmic input as well as titration recommendations from the algorithm residing within it. Glucose data may be calculated and supplied directly within the mobile application or supplied to the mobile application from an outside source. In a web-based application, a care provider could track a user's glycemic control, and insulin dosing habits if available, via reports as well as receive titration recommendations for review and approval upstream of any patient notification. In a dual application system, a care provider could “approve” a dose change recommendation within the web application. This action would then immediately push a notification to the user mobile app of a new dose amount. In some embodiments, this approval by the HCP could be required before outputting the recommended action to the user. In other embodiments, pre-approval by the HCP may not be required before outputting the recommended action to the user.

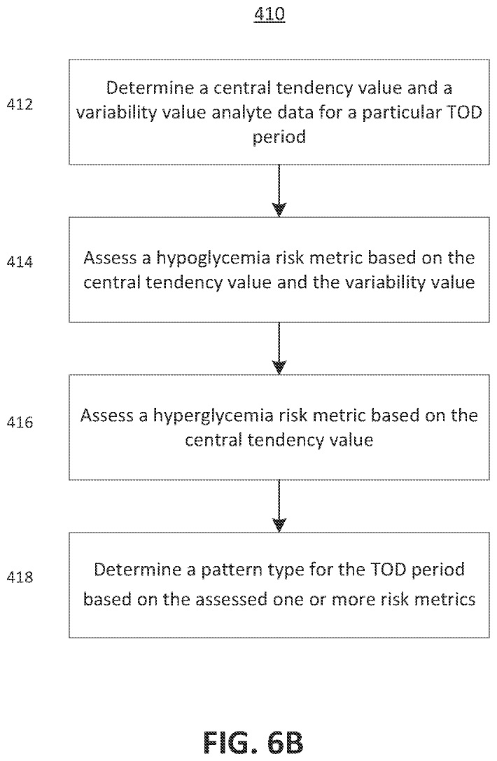

Thus, there are many different embodiments pertaining to the number and type of electronic devices that are used in storing, executing, and presenting the DGA to a user. With respect to presentation to the user, the device that is configured to implement this capability will be referred to herein as a user interface device (UID) 200. FIG. 5 is a block diagram depicting an example embodiment of UID 200. In this embodiment, UID 200 includes a housing 201 that is coupled with a user interface 202. The user interface 202 is capable of outputting information to the user and receiving input or information from the user. In some embodiments, the user interface 202 is a touchscreen. As shown here, the user interface 202 includes a display 204, that may be a touchscreen, and an input component 206 (e.g., a button, actuator, touch sensitive switch, capacitive switch, pressure sensitive switch, jog wheel, microphone, touch pad, soft keys, keyboard, or the like).

Many of the devices described herein can be implemented as UID 200. For example, display device 120 will, in many embodiments, be used as UID 200. In some embodiments, MDD 152 can be implemented as UID 200. In embodiments where SCD 102 includes a user interface, then SCD 102 can be implemented as UID 200. Computer system 170 can also be implemented as UID 200.

### Purpose

The Dose Guidance System (DGS 100) leverages glucose data and additional data, such as typical meal and bed times, to titrate insulin doses such as basal insulin doses. The DGS includes an application, e.g., a mobile application based on a smart-phone, integrated with a connected insulin pen and continuous glucose sensor to improve therapy management for insulin-intensive people with diabetes (PWDs) on basal insulin or multiple daily injections (MDI).

Continuous glucose data in various forms (real-time, scan, historic, and streaming) as well as insulin data, may serve as inputs to perform all three functions above. The DGS 100 may receive the glucose data by different means and in various forms, including scan, historic, and streaming. Scanned data, including latest glucose values and trend values, may have been retrieved by the user on demand. Historic glucose data may be generated by components of the DGS, which may generate and record glucose and trend values at a regular interval, e.g., every 15 minutes. Past historic data may be retrieved by the user with a scan. Streaming data may include glucose and trend values that are generated and recorded at a regular interval, e.g., every minute, and automatically sent to the DGA. Sensor data may have regular intervals of one minute, 5 minutes, 10 minutes or 15 minutes between readings.

The system may also receive insulin data from multiple sources. Insulin data may be manually logged or transferred from an MDD 152, e.g., insulin pen. The glucose data and insulin data may be transferred through any known means, e.g., wireless communication technology such as Bluetooth or NFC.

Other aspects of dose guidance systems are described in US 2021/0050085 and US 2022/0249779, which are hereby expressly incorporated by reference in their entireties for all purposes.

### Glucose Pattern Analysis for Insulin Dosing Therapies

Example embodiments of methods for determining insulin titrations will now be described. The algorithm may have two classes of inputs: (1) glucose data and (2) typical times for meals and bedtime. The outputs will be glucose patterns and hypoglycemic risk for each time-of-day period defined below. If appropriate, these outputs will then map to a basal insulin titration recommendation to be presented within the application.

The DGA can request that typical times for meals and bedtimes be inputted or estimated to define Time-of-Day (TOD) periods that can be used to analyze the analyte data. The TOD periods may include overnight, post-breakfast, post-lunch and post-dinner periods. Daily meal and bedtimes may be either fixed or user-configured and may be entered by either the patient or care provider. Bedtime may be entered by the user or estimated from user-entered mealtimes. These entered/calculated times may define time-of-day (TOD) periods that will be used to analyze the glucose data. Demarcation according to mealtimes and sleep are valuable in the context of insulin titration as the post-meal glucose and fasting overnight windows are key indicators of drug efficacy. In other embodiments, TOD periods can be defined in other ways and do not need to be strictly defined as above for the algorithm to function.

In some embodiments, the DGA may also receive additional input from the user such as the user's weight, insulin dose timing and amounts, and meal and exercise logs. These extra inputs could be achieved either manually through logging in a mobile application or in a hands-off fashion via communication from a connected device (e.g., a Bluetooth-enabled insulin pen or pen cap, activity monitors, etc.) to the application.

Glucose values may be collected over multiple days and binned according to the TOD period in which their timestamps fall. Once sufficient data have been gathered in all TOD periods, metrics for each period may be calculated to quantify the user's glucose control during each period.

One or more metrics can be determined for the different TOD periods. These one or more metrics may be used by the DGA to determine a likelihood of low glucose (LLG) metric and the median glucose can be used to quantify the degree of hypoglycemia risk and hyperglycemia risk, respectively. Those metrics may be any glucose-derived metric (e.g., average glucose during the window, time within a target range, a combination of the two), median glucose, variability below the median (median less 10¹ percentile), and the likelihood of low glucose (LLG; defined in other disclosures) are considered. The four TOD periods may be defined as overnight, post-breakfast, post-lunch, and post-dinner. The metrics calculated within each period may then mapped to a glucose period pattern.

U.S. Patent Publ. No. 2018/0188400 (the '400 publication), which is incorporated by reference herein for all purposes, describes examples of an implementation for deriving and determining risk metrics that can be utilized in glucose pattern analysis (GPA) for the DGA embodiments. This implementation, among other things, utilizes central tendency (e.g., mean, median, etc.) and variability data from the multi-day period to determine a risk metric corresponding to a degree of hypoglycemia risk (“hypo risk”). This implementation is summarized herein, and a more exhaustive description of the implementation and variations therefrom can be obtained by reference to the '400 publication.

Alternatives to the implementation described in the '400 publication are set forth in U.S. Patent Publ. No 2014/0350369, which is also incorporated by reference herein for all purposes. For example, instead of using median and variability, the method could employ any two statistical measures that define a distribution of data. As described in the '369 publication, the statistical measures could be based on a glucose target range (e.g., G_{LOW}=70 mg/dL and G_{HIGH}=140 mg/dL). Common measures related to the target range are time in the target range (TIR), time above target (t_{AT}), and time below target (t_{BT}). If the glucose data is modeled as a distribution (e.g., a gamma distribution), for predefined thresholds G_{LOW} and G_{HIGH}, then t_{AT} and t_{BT} can be calculated. For the thresholds, an algorithm can also define t_{BT_HYPO} in which if exceeded by t_{BT}, then the patient may be determined to be a high hypoglycemia risk. For example, high hypoglycemia risk can be defined as whenever t_{BT} is greater than 5% for G_{LOW}=70 mg/dL. Similarly, a metric t_{AT_HYPER} can be defined in which if exceed by t_{AT}, then the patient can be determined to be at high hyperglycemia risk. The degree of hypoglycemia and hyperglycemia risk can be adjusted by adjusting either G_{LOW} or t_{BT_HYPO}, or G_{HIGH} or t_{AT_HYPER}, respectively. Any two of the three measures, TIR, t_{BT}, and t_{AT} can be used to define a control grid. These alternatives (and others) can be used to determine risk metrics for the DGA embodiments described herein.

The DGA embodiments described herein can operate based on a quantitative assessment of the user's analyte data during a TOD period. This quantitative assessment can be performed in various ways. For example, the embodiments described herein can assess the analyte data over a multi-day period to determine one or more metrics that are descriptive of relevant risks exhibited by that analyte data for a corresponding TOD. These metrics can then be used to classify the analyte data from the TOD period as one of multiple patterns. For example, these patterns can be indicative of a common or prevalent glucose behavior or trend for that TOD. Any number of two or more patterns can be utilized by the DGA embodiments. For ease of reference herein, these patterns are referred to as glucose pattern types and the embodiments described herein will make reference to an implementation utilizing three glucose pattern types (e.g., a low pattern, a high/low pattern, and a high pattern), although other implementations may utilize only two types or more than three types, and those types may differ from those described herein.

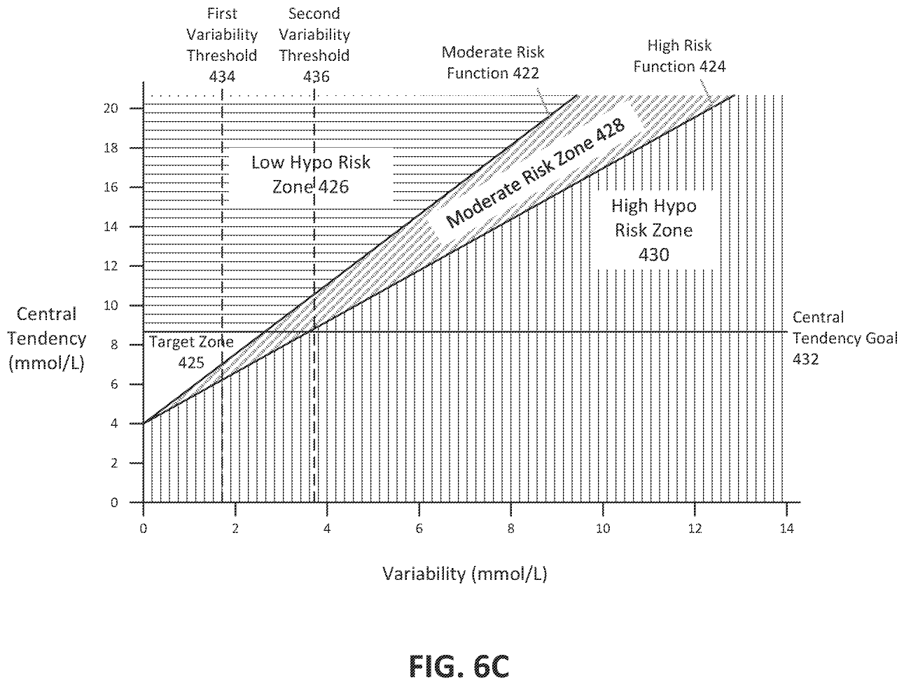

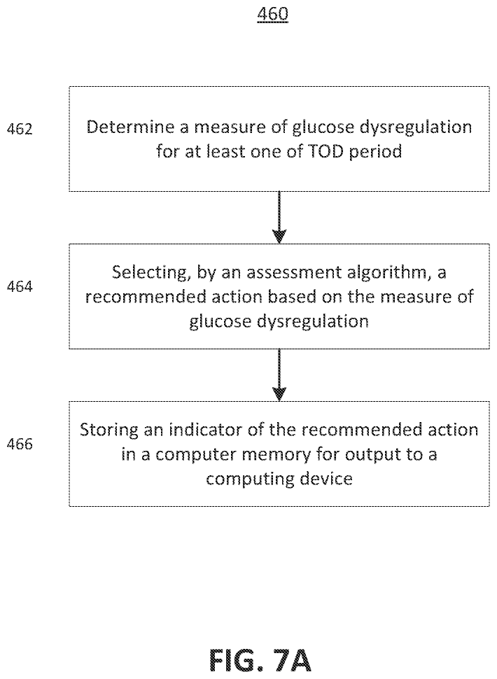

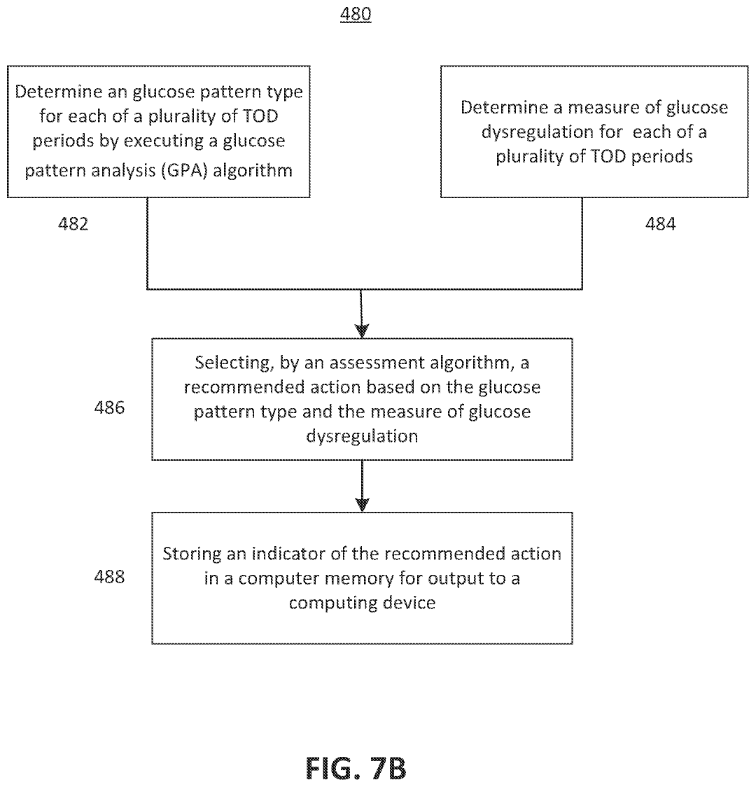

Using basal insulin doses for example, titration assessment can begin. For each TOD period (overnight, post-breakfast, post-lunch and post-dinner), the DGA can map the two metrics described above (LLG and median glucose) to the four logic “pattern” variables per the GPA method described below. FIG. 6A shows operations of an example method 400 by a DGA for assessing a basal titration. The method 400 can include, at 402, determining, by a DGA, an analyte pattern type for each TOD period of a plurality of TOD periods by executing a glucose pattern analysis (GPA) algorithm that receives, as input, time-correlated analyte data originating from a sensor control device worn by a patient over an analysis period. The method 400 can further include, at 404, selecting by the DGA executing a recommendation algorithm, a basal dosing recommendation based on the analyte pattern types determined for the plurality of TOD periods. The method 400 can further include, at 406, storing, by the DGA an indicator of the recommended action in a computer memory for output to a computing device, such as a UID 200 or an MDD 152 administering medication. A UID 200 can use the indicator of the recommended action to control a user interface, for example, by causing a human-readable expression of the indicator to appear on a display, or by generating an audio output expressing the indicator in a human language. An MDD 152 can use the indicator to adjust or maintain a next relevant dose administration. Further details of the method 400 are described below.

FIG. 6B is a flowchart depicting an example embodiment of a GPA method 410 that can be implemented as the GPA algorithm referenced in 402. Method 410 can be performed for a particular TOD period that can be an entire day (e.g., a 24 hour period), or a portion of a day that is delineated by time blocks (e.g., three 8 hour periods) or the user's activities (e.g., meals, exercise, sleep, etc.). In many embodiments, multiple TOD periods can correspond to meals (e.g., post-breakfast, post-lunch, post-dinner) and sleep (e.g., overnight). These TOD periods can correspond to fixed times of the day where the activity would normally occur (e.g., post-breakfast from 5 am to 10 am), where such time blocks can be set by the user, or can be contingent on the meal or activity actually having been performed as determined by the automated detection of the meal or activity, or by a user indication of such (e.g., with UID 200).

A DGA can perform the method 410 independently for each TOD period to arrive at a separate pattern assessment for that period. At 412, the DGA can determine a central tendency value and a variability value from the user's analyte data for the particular TOD period. The user's analyte data may be available from the user's own records or those of the user's healthcare professional, or the user's analyte data may have been collected by DGS 100, for example. The analyte data preferably spans a multi-day period (e.g., two days, two weeks, one month, etc.) such that sufficient data exists within the TOD period to make a reliable determination. In other embodiments, the method can be performed in real-time on limited data. The DGA can use any type of central tendency metric that correlates to a central tendency of the data including, but not limited to, a median or mean value. Any desired variability metric can also be used including, but not limited to, variability ranges that span the entire data set (e.g., from the minimal value to the maximum value), variability ranges that span a majority of the data but less than the entire data set so as to lessen the significance of outliers (e.g., from the 90^{th} percentile to the 10^{th} percentile, from the 75^{th} percentile to the 25^{th} percentile), or variability ranges that target a specific asymmetrical range (e.g., low range variability, which can span a range, e.g., from or in proximity with the central tendency value to a lower value of data, e.g., the 25^{th} percentile, the 10^{th} percentile, or the minimal value). The selection of the metrics to represent the central tendency and variability can vary based on the implementation.

At 414, the DGA can assess a risk of hypoglycemia (“hypo risk”) metric based on the central tendency value and the variability value. One such methodology for determining hypo risk is described with respect to FIG. 6C, showing an example embodiment of a framework for determining hypo risk and other metrics. While FIG. 6C is intended to convey the framework to the reader, however, this framework can be implemented electronically in numerous different ways, such as with a software algorithm (e.g., a mathematical formula, a set of if-else statements, etc.), a lookup table, firmware, a combination thereof, or otherwise.

FIG. 6C is a graph of central tendency versus variability (e.g., low range variability) that can be used to evaluate or identify a region or zone that holds or corresponds to a determined central tendency and variability data pair for a particular TOD. Any number of two or more zones can be used. In this embodiment the data pair can correspond to a target zone 425 or one of three hypo risk zones: a low zone 426, a moderate zone 428, or a high zone 430. A first hypo risk function (e.g., a curved or linear boundary), referred to as moderate risk function 422, differentiates between low zone 426 and moderate zone 428. A second hypo risk function, referred to as high risk function 424, differentiates between moderate zone 428 and high zone 430. The central tendency and variability data pair can be evaluated against or compared to the zones to determine a hypo risk metric for the corresponding TOD period.

The hypo risk functions 422 and 424 can be implemented in the DGA explicitly as a mathematical function (e.g., a polynomial) or can be implemented implicitly, such as by defining each zone by the pairs it contains, use of a lookup table, set of if-else statements, threshold comparisons, or otherwise. The hypo risk functions 422 and 424 can be preloaded into the DGA, or can be downloaded from trusted computer system 480, or can be set by another party such as the HCP. Once implemented in the DGA, the hypo risk functions 422 and 424 can be treated as fixed or can be adjusted by the user or HCP. Example methodologies for determining the hypo risk function are described in the '400 publication.

At 416, the DGA can assess a hyperglycemia risk metric (“hyper risk”) based on the central tendency value. In this embodiment, the hyper risk can be evaluated by comparison of the central tendency value for the particular TOD period to a central tendency goal or threshold 432. The magnitude and/or sign of the difference of the central tendency value from the goal 432 can identify the amount of hyper risk. For example, a low hyper risk can be present if the central tendency value is less than the goal 432 (e.g., a negative value). A moderate hyper risk can be present if the central tendency value exceeds the goal 432 (e.g., a positive value) by less than a threshold amount (e.g., 5 percent, 10 percent, etc.). A high hyper risk can be present if the central tendency value exceeds the goal 432 by a value greater than the threshold amount. The use of three discrete groupings for hyper risk (e.g., low, moderate, high) is an example and any number of two or more groupings can be used.

In other embodiments, the DGA can assess a hyperglycemia risk metric, at 416, before assessing a hypoglycemia risk, at 414. Alternatively, in another embodiment, the assessments of hypoglycemia risk, at 414, and hyperglycemia risk, at 416, can be done in parallel at the same time.

Other metrics such as variability risk can also be assessed. For example, a variability value less than a first variability threshold 434 can be indicative of a low variability risk, a variability value greater than the first variability threshold 434 and less than a second variability threshold 436 can be indicative of a moderate variability risk, and a variability value greater than the second variability threshold 436 can be indicative of a high variability risk. Again, the use of three discrete groupings for variability risk is an example. The DGA can use any number of two or more groupings.

At step 418, the DGA can determine a pattern type for the TOD period based on the assessed one or more risk metrics. In one example embodiment, pattern determination can be assessed with the hypo risk metrics and the hyper risk metric. If the hypo risk metric is high, then the pattern can be set as a low pattern (or “Lows” pattern). Otherwise, if the hypo risk is moderate and the hyper risk is either high or moderate then the pattern can be set as a high/low (or moderate) pattern (or “Lows with Some Highs” or “Highs with Some Lows”). Otherwise, if the hyper risk is high or moderate and the hypo-risk is low, then the pattern can be set as a high pattern (or “Highs” pattern). If both the hyper risk and hypo risk are low, then the pattern identified can be No Problem (e.g., an “OK” message is displayed our outputted) (or “no pattern”).

An exemplary method for determining a glucose period pattern is presented below in pseudo-code.

```
if LLG is high
  then period pattern = LOW
elseif (median glucose is moderate or high AND LLG is moderate)
  then period pattern = HIGH/LOW
elseif (median glucose is moderate or high AND LLG is low)
  then period pattern = HIGH
elseif (median glucose is low and LLG is low or moderate)
  then period pattern = NONE
end
```

Thus, method 410 is one example of how the DGA can output one of multiple pattern types for each TOD period. The number of pattern types in the pattern types themselves can vary from those described in this embodiment (e.g., low, high/low, high). Once pattern type for the TOD period has been determined, the DGA can store an indicator of the pattern type in a memory location for use in determining a titration recommendation. Referring again to FIG. 6A, the DGA can proceed, at 404, to determine a titration recommendation once completing the GPA for each relevant TOD period.

Based on the pattern analysis, the DGA may make a recommendation to adjust a basal insulin dose amount. For instance, the DGA may recommend to increase a basal insulin dose amount when a HIGH pattern is detected and there is no hypo risk is determined in any other TOD period. Alternatively, the DGA may recommend to decrease basal insulin when a LOW pattern in at least one TOD period is detected.

### Glucose Dysregulation Analysis

In an alternative method, the DGA can determine a measurement of glucose dysregulation to determine a recommended insulin titration. The application may contain an algorithm that will interrogate glucose data to determine patterns of glucose dysregulation and suggest subsequent corrective therapeutic action.

In some embodiments, the DGA may count instances of glucose dysregulation. Glucose dysregulation may be determined by different methods. For example, in one embodiment, glucose dysregulation may be a count of instances where a glucose signal crosses above or below a threshold value, such as above 180 mg/dL or below 70 mg/dL. In another embodiment, glucose dysregulation may be a duration of time above or below a threshold value. In another embodiment, glucose dysregulation may be an area over or under a threshold value. The threshold value may either be fixed within the algorithm, user-configured, or configured by a care provider (e.g., HCP, parent, or guardian) of the user. In another embodiment, the threshold value may be the same as high or low glucose alarm thresholds within a user's continuous glucose monitor and associated mobile application, such that the counting events map to low or high glucose alarm instances presented to the user. Such a determination based on area would represent a combination of magnitude and time duration. Dysregulation instances may be counted and binned according to the TOD period or within another time window (e.g., instances in a day or instances in a week). In this way, the threshold value may be represented as the rate of dysregulation instances within a time window instead of just the number of instances. If the user's rate of dysregulation instances crosses the threshold, a titration recommendation may be made by the DGA. In another embodiment, glucose dysregulation may be determined by the number of days with at least a minimum number of instances of glucose dysregulation described above. The frequency of glucose dysregulation can be used as part of the insulin dose titration algorithm. Herein, the two types of glucose dysregulation may be referred to as a High Event for when glucose is above a threshold, and a Low Event for when glucose is below a threshold. In one embodiment, a High Event may be defined as the instant that the glucose crosses the High Threshold, e.g., 180 mg/dL. A Low Event may be defined as the instant that the glucose crosses the Low Threshold. In other embodiments, a High Event may be defined as when a High Glucose Alarm is first asserted, and a Low Event may be defined as when a Low Glucose Alarm is first asserted. In one embodiment, the insulin titration algorithm may include logic that depends on the frequency of Low Events and/or High Events, where the frequency can be determined in a number of ways. For instance, for basal insulin titration, the dose titration algorithm may depend on the count of the number of days with a Low Event, in the past seven days. For multiple-daily-injection insulin titration, the dose titration algorithm may depend on the count of the number of days with a Low Event and/or a High Event that is initiated during the TOD related to the particular insulin dose.

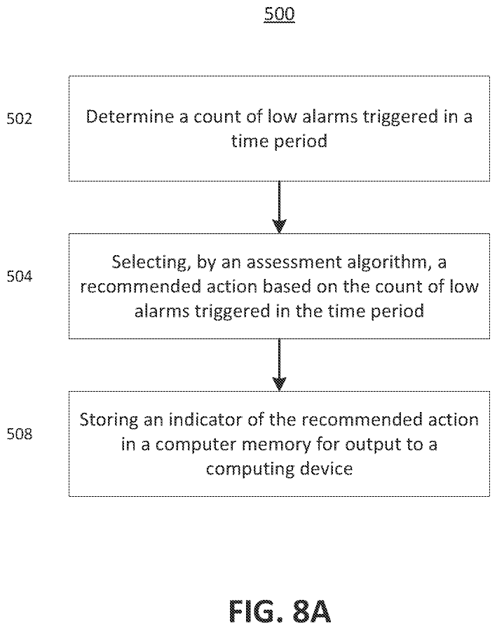

FIG. 7A shows operations of an example method 460 by a DGA for assessing a basal titration. The method 460 can include, at 462, determining, by a DGA, a measure of glucose dysregulation for at least one TOD period by executing an algorithm that receives, as input, time-correlated analyte data originating from a sensor control device worn by a patient over an analysis period. The method 460 can further include, at 464, selecting by the DGA executing a recommendation algorithm, recommended action based on the measure of glucose dysregulation for the at least one TOD period. The method 460 can further include, at 466, storing, by the DGA, an indicator of the recommended action in a computer memory for output to a computing device, such as a UID 200 or an MDD 152 administering medication. A UID 200 can use the indicator of the recommended action to control a user interface, for example, by causing a human-readable expression of the indicator to appear on a display, or by generating an audio output expressing the indicator in a human language. An MDD 152 can use the indicator to adjust or maintain a next relevant dose administration.

In some embodiments, as seen in FIG. 7B, the DGA may perform a glucose dysregulation analysis in addition to the glucose pattern analysis to determine a recommended insulin titration. The method 480 can include, at 482, determining, by a DGA, a glucose pattern type for each TOD period of a plurality of TOD periods by executing a glucose pattern analysis (GPA) algorithm that receives, as input, time-correlated analyte data originating from a sensor control device worn by a patient over an analysis period. The method 480 can further include, at 484, determining, by a DGA, a measure of glucose dysregulation for each TOD period of a plurality of TOD periods by executing an algorithm that receives, as input, time-correlated analyte data originating from a sensor control device worn by a patient over an analysis period. The method 480 can further include, at 486, selecting by the DGA executing a recommendation algorithm, a recommended action based on the glucose pattern type and the measure of glucose dysregulation for the at least one TOD period. The method 480 can further include, at step 488, storing, by the DGA, an indicator of the recommended action in a computer memory for output to a computing device, such as a UID 200 or an MDD 152 administering medication. A UID 200 can use the indicator of the recommended action to control a user interface, for example, by causing a human-readable expression of the indicator to appear on a display, or by generating an audio output expressing the indicator in a human language. An MDD 152 can use the indicator to adjust or maintain a next relevant dose administration.

Determining a recommended action by using both a glucose pattern analysis algorithm and a glucose dysregulation algorithm has many advantages. The glucose dysregulation analysis complements a pattern-based method by increasing algorithmic sensitivity to glucose dysregulation. Moreover, because a pattern-based method may require greater than five days of data to identify dysregulation, a glucose dysregulation counting method could allow for faster response time to detect glaring instances of poor control before sufficient data have been gathered for pattern analysis. Such a counting method may also be extendable beyond basal insulin titration and could be applied to titrating any medication that alters analyte levels. One such extension is the titration of rapid acting prandial insulin. Additionally, if multiple administrations of a given dose mealtime dose amount confer multiple instances of glucose dysregulation, this dysregulation counting method could be employed to complement pattern-based titration methods and improve responsiveness of a medication titration algorithm.

### Low Alarm Frequency Analysis

Tracking CGM low alarms in medication titration algorithms may also be used to identify and correct medication-induced hypoglycemia by decreasing overly aggressive drug dosing.

In some embodiments, the DGA may count a number of low alarms triggered in a time period, such as 1 week. If the number of low alarms triggered is above a threshold value (e.g., 1), the LOW pattern discussed above with respect to the GPA analysis may be implemented and a recommended action may include a recommendation to decrease a dose amount, e.g., a decrease a recommended basal or bolus dose amount. The threshold value (i.e., the amount of allowable low alarms per time period) may be adjusted as an input to make the titration algorithm more or less aggressive, or to reflect a patient's tolerance of low alarms.

In some embodiments, the DGS may maintain a counter that counts the number of low alarms that occur during a time period, such as a TOD period. This counter may be checked against a threshold value, e.g., 1, 2, 3, 4, or 5. If the threshold value is reached or exceeded, then a LOW pattern may be inputted and a recommended action may be determined.

Determining a recommended action by including a low alarm analysis has many advantages. The rationale behind this approach is that when patients are using an unblinded CGM, and when they are aware that their glucose is low, they often “treat” their low glucose by administering carbohydrates. The result is that often low patterns cannot be detected when they normally would for a blinded CGM system. If the titration algorithm does not take low alarm frequency into account, then the algorithm may continue to increase the dose indefinitely, up to the point when low alarm frequency becomes intolerable to the patient. Even more concerning is that if the patient, who is aggressively dosing and has a high low alarm frequency, discontinues the use of unblinded CGM for any reason, they will no longer be able to effectively manage their low glucose occurrences, and may experience substantial hypoglycemia.

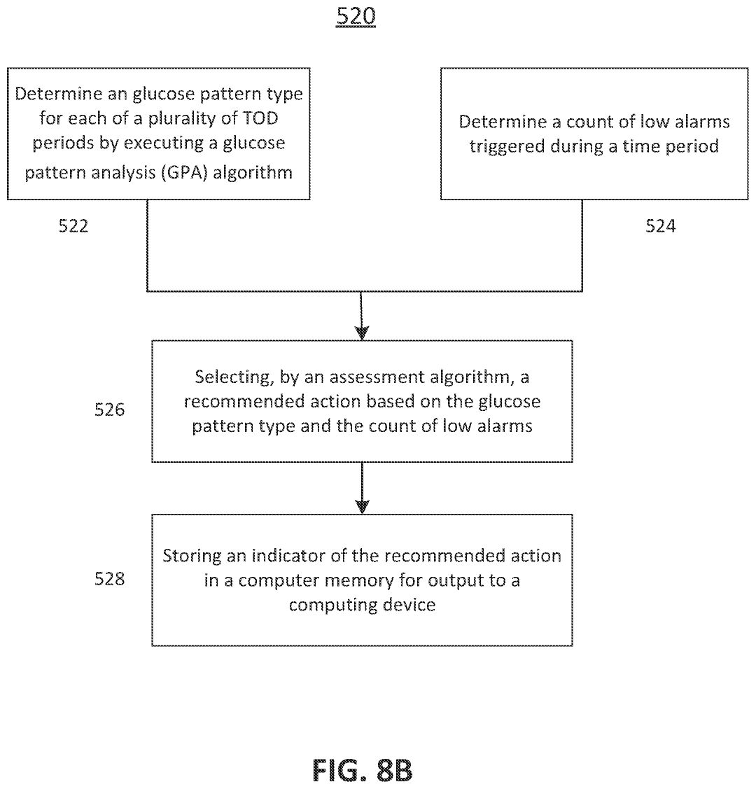

FIG. 8A shows operations of an example method 500 by a DGA for assessing a basal titration. The method 500 can include, at 502, determining a count of low alarms triggered in a time period. The algorithm may receive, as input, time-correlated analyte data originating from a sensor control device worn by a patient over an analysis period, and/or may also receive a log or count of low alarms or events detected that satisfy a low alarm condition. The low alarm condition may be user configured to activate once a glucose reading crosses below a set value (e.g., 70 mg/dL) and may remain ON until such point that the glucose rises above the set value or the set value plus some buffer. Note that other definitions of a low alarm may apply here, as are well known to those skilled in the art. The method 500 can further include, at step 504, selecting a recommended action based on the count of low alarms triggered in the time period. The method 500 can further include, at step 508, storing an indicator of the recommended action in a computer memory for output to a computing device, such as a UID 200 or an MDD 152 administering medication. A UID 200 can use the indicator of the recommended action to control a user interface, for example, by causing a human-readable expression of the indicator to appear on a display, or by generating an audio output expressing the indicator in a human language. An MDD 152 can use the indicator to adjust or maintain a next relevant dose administration.

In some embodiments, as seen in FIG. 8B, the DGA may perform a low alarm frequency analysis in addition to the glucose pattern analysis to determine a recommended insulin titration. The method 520 may include, at step 522, determining a glucose pattern type for each TOD period of a plurality of TOD periods. The method 520 can further include, at step 524, determining a count of low alarms triggered in a time period. The algorithm may receive, as input, time-correlated analyte data originating from a sensor control device worn by a patient over an analysis period, and/or may also receive a log or count of low alarms or events detected that satisfy a low alarm condition. The method 520 can further include, at step 526, selecting a recommended action based on the glucose pattern type and the count of low alarms triggered in a time period. The method 520 can further include, at step 528, storing an indicator of the recommended action in a computer memory for output to a computing device, such as a UID 200 or an MDD 152 administering medication. A UID 200 can use the indicator of the recommended action to control a user interface, for example, by causing a human-readable expression of the indicator to appear on a display, or by generating an audio output expressing the indicator in a human language. An MDD 152 can use the indicator to adjust or maintain a next relevant dose administration.

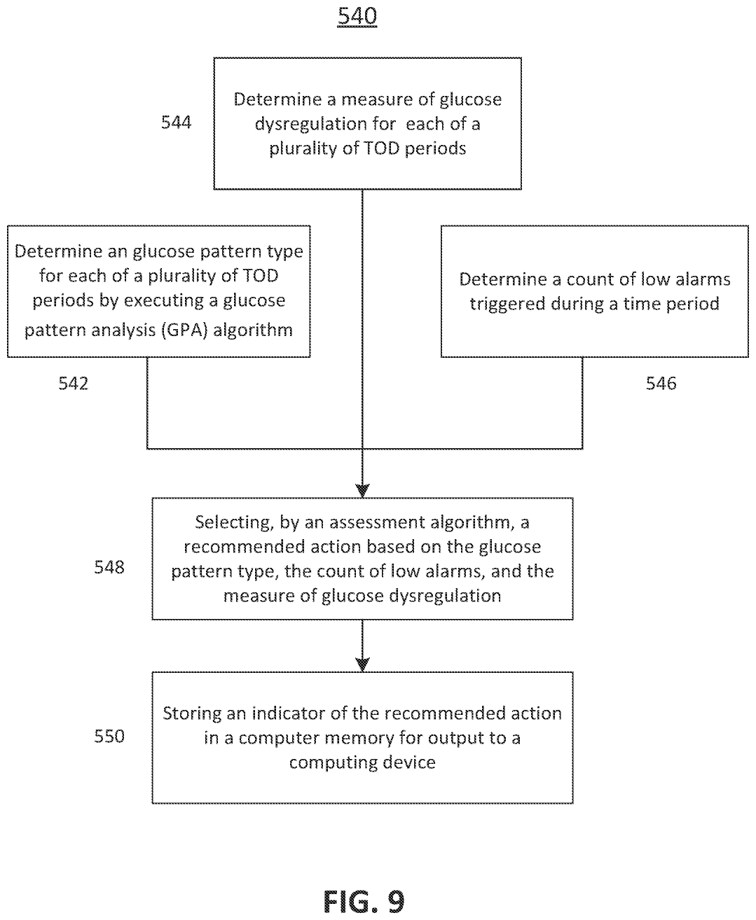

In some embodiments, as seen in FIG. 9, the DGA may perform a low alarm frequency analysis and a glucose dysregulation analysis in addition to the glucose pattern analysis to determine a recommended insulin titration. The method 540 can include, at step 542, determining a glucose pattern type for each TOD period of a plurality of TOD periods. The method 540 can further include, at step 544, determining a measure of glucose dysregulation for each TOD period of a plurality of TOD periods by executing an algorithm that receives, as input, time-correlated analyte data originating from a sensor control device worn by a patient over an analysis period. The method 540 can further include, at step 546, determining a count of low alarms triggered in a time period. The algorithm may receive, as input, time-correlated analyte data originating from a sensor control device worn by a patient over an analysis period, and/or may also receive a log or count of low alarms or events detected that satisfy a low alarm condition. The method 540 can further include, at step 548, selecting a recommended action based on the glucose pattern type, the count of low alarms triggered in a time period, and the measure of glucose dysregulation. The method 540 can further include, at step 548, storing an indicator of the recommended action in a computer memory for output to a computing device, such as a UID 200 or an MDD 152 administering medication. A UID 200 can use the indicator of the recommended action to control a user interface, for example, by causing a human-readable expression of the indicator to appear on a display, or by generating an audio output expressing the indicator in a human language. An MDD 152 can use the indicator to adjust or maintain a next relevant dose administration.

The basal dose titration algorithm may have the following rules:
If any TOD period has a Low Pattern OR the Frequency of Low Events exceeds 2 Days with Low Event in the past seven days, then decrease the basal dose;
Else if any TOD period has a High Pattern AND no TOD period has Moderate Hypo Risk, then increase the basal dose;
Else if any TOD period has a High Pattern, then provide a notification to indicate that other therapy modifications may be needed;
Else, provide a notification that the patient has good glucose control.

For MDI titration methods, the Low and High Event frequency metrics may likewise be used in conjunction with the pattern analysis to determine if the dose associated with a TOD should be increased or decreased. For instance, for a particular TOD, if the pattern is a LOW pattern OR if the Low Event frequency for that TOD period exceeds a threshold, then that dose will be decreased.

Note that Low Event frequency and High Event frequency may be incorporated into any form of dose titration. For instance, there are currently methods that are publicly known for titrating basal insulin based on standard time-in-range (TIR), time-above-range (TAR), and time-below-range (TBR) glucose metrics. Low Event frequency may be included in the titration logic, such that the dose is decreased if the TBR threshold is exceeded OR if the Low Event frequency threshold is exceeded. Else the dose is increased if the TAR threshold is exceeded OR if the High Event frequency threshold is exceeded. A similar coupling of logic may be made for MDI dose titration algorithms.

### Optimal Control

Optimal control may be detected when titrations no longer trend in a specific direction. For instance, optimal control may be determined to have been reached by the system when the same dose is repeated for a minimum amount of times in a time period or in a number of titration changes, e.g., three repeats in 8 titration changes. Alternatively, optimal control may be determined to have been reached by the system when the outputted recommended change in dose alternates for a consecutive number of times, e.g., alternating up, down, up or alternating down, up, down. The consecutive number of alternative recommended changes may be at least 3, alternatively at least 4, alternatively at least 5, alternatively at least 6.

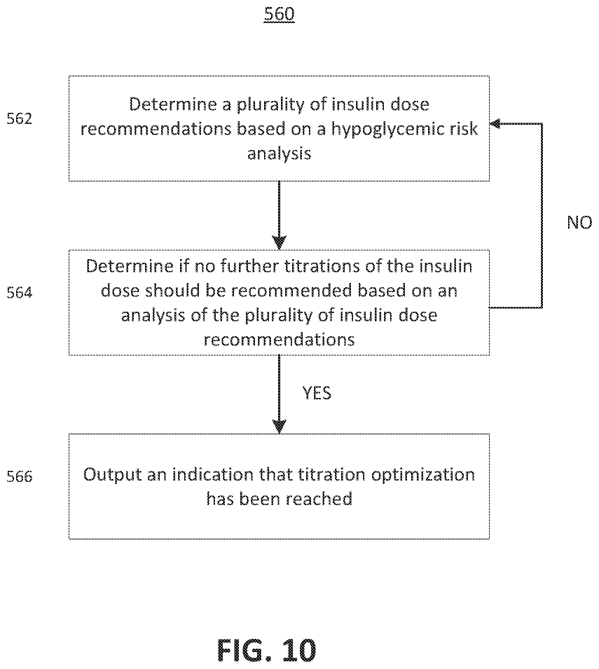

FIG. 10 illustrates an exemplary method for determining if optimal control has been reached and no further titration recommendations will be made. In method 560, at step 562, determining a plurality of insulin dose recommendations based on a hypoglycemic risk analysis. The hypoglycemic risk analysis can be based on known methods, including the glucose pattern analysis, glucose dysregulation analysis, low alarm frequency analysis, and combinations thereof, as discussed elsewhere herein. At step 564, the system may determine if no further titrations of the insulin dose should be recommended based on an analysis of the plurality of insulin dose recommendations. If it is determined that further titrations should still be made, the method may return to step 562. If it is determined that no further titrations should be made, then at step 566, an indication that titration optimization has been reached may be outputted.

In other embodiments, measures of optimal glucose control may be based on analysis of a central tendency, e.g., mean or median, a variability measure like variance, or the spread of percentile values.

Once the system has determined that optimal control has been reached, a more elaborate optimization detection methods may be utilized.

### Recommended Actions

The DGA may store and/or output a variety of recommended actions depending on the analysis. In one embodiment, the DGA may store and/or output a recommended change in a dose as a percentage of a current dose. Alternatively, the DGA may store and/or output a recommended change in a dose in units of insulin. In another embodiment, the DGA may store and/or output a recommended dose in units. The titration amounts may vary according to the magnitude of dysregulation, such that larger changes may be recommended when a user's glucose metrics are far from optimal.

In some embodiments, the DGA may recommend adding a new medication class to the user's therapy. For example, a person may present both a high median glucose, which would suggest they are in need of a basal insulin dose increase, in conjunction with high variability below the median. This high variability below the median means that any basal insulin dose increase would likely induce instances of hypoglycemia. As such, the basal dose cannot be titrated despite the high median glucose. Conversely, the dose should not be decreased because that will induce a rise in median glucose. In situations where basal insulin can no longer be titrated due to the presence of high glucose variability, the system may recommend an additional glucose-regulating medication. The system may also request that the user input their weight. With the user's weight, the system could track the weight normalized basal insulin dose (in units of U/kg). Many healthcare providers believe that a person's basal insulin dose should never exceed 0.5 U/kg. If basal dose titrations exceed such an upper threshold, this too could be a useful criterion for recommending initiation of a new glucose-regulating therapy.

The DGA may determine that optimal control of glucose is occurring. Optimal control may be detected when titrations no longer trend in a specific direction. Optimal control may also be detected when the same dose is repeated some number (say three times in eight titration changes), where the dose is output after alternating up, down, up titration (or down, up, down). More elaborate optimization detection methods may be contemplated. Measures of optimal glucose control may be according to a central tendency like mean or median or a variability measure like variance or the spread of percentile values.

Different outputs may be displayed depending on the recipient. Outputs to the HCP may include recommending the next dosing amount change, that the maximum recommended dose has been reached, that the titration optimization has been reached and the patient is in good glucose control, or that titration optimization has been reached and the patient remains in poor glucose control indicating that therapy escalation may be required. Outputs to the user may include a recommended change in the dose amount.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. Thus, the foregoing description of specific embodiments of the disclosed subject matter has been presented for purposes of illustration and description. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents. Furthermore, any features, functions, steps, or elements of the embodiments may be recited in or added to the claims, as well as negative limitations that define the inventive scope of the claims by features, functions, steps, or elements that are not within that scope.

In many embodiments, a method for determining a titration for basal insulin dosing includes the steps of: determining, by at least one processor, an analyte pattern type for each of a plurality of time of day (TOD) periods by executing a pattern analysis algorithm that receives as input time-correlated analyte data of a patient taken over an analysis period; selecting, by the at least one processor executing a recommendation algorithm, a recommended action based on the analyte pattern type; and storing, by the at least one processor, an indicator of the recommended action in a computer memory for output.

In some embodiments, the recommended action is a basal dosing recommendation.

In some embodiments, the method further includes the step of determining, by at least one processor, a measure of glucose dysregulation for the plurality of TOD periods, wherein the recommended action is selected based at least on the analyte pattern type and the measure of glucose dysregulation. In some embodiments, the measure of glucose dysregulation is determined by determining a number of times an analyte level crosses above or below a threshold crossing value in a time period. In some embodiments, the measure of glucose dysregulation is determined by determining a duration of time above or below a threshold value in a time period. In some embodiments, the measure of glucose dysregulation is determined by determining an area over or under a threshold area value in a time period. In some embodiments, the measure of glucose dysregulation is determined by determining a number of days in a time period with a minimum number of instances of glucose dysregulation.

In some embodiments, the method further includes the step of determining, by at least one processor, a frequency of a low glucose alarm within a time period, wherein the recommended action is selected based at least on the analyte pattern type and the frequency of the low glucose alarm. In some embodiments, the step of determining, by the at least one processor, the frequency of the low glucose alarm within the time period comprises determining if the low glucose alarm was triggered more than a threshold number of times.

In some embodiments, the method further includes the step of outputting, by the at least one processor, the recommended action. In some embodiments, the recommended action is a change to a next dosing recommendation. In some embodiments, the change is a percentage of a current dose. In some embodiments, the change is a value of a dose in units. In some embodiments, the recommended action is a recommendation to add a new medication. In some embodiments, the recommended action is outputted to an HCP. In some embodiments, the recommended action is outputted to a user. In some embodiments, the recommended action is an indication that a maximum recommended dose has been reached.

In some embodiments, the recommended action is an indication that an optimization in titration has been reached. In some embodiments, the recommended action further states that a user is in good glucose control. In some embodiments, the recommended action further states that a user remains in poor glucose control. In some embodiments, the recommended action further indicates that therapy escalation may be required.

In some embodiments, selecting the recommended action is based on the analyte pattern type and additional input. In some embodiments, the additional input comprises a user's weight. In some embodiments, the additional input comprises insulin dose data comprising dose amounts and corresponding times of administration. In some embodiments, the additional input comprises meal logs. In some embodiments, the additional input comprises exercise logs.

In many embodiments, a system for determining a recommended medication dose includes an input configured to receive time-correlated analyte data of a patient taken over an analysis period; one or more processors coupled with the input and a memory storing instructions, wherein the instructions, when executed by the one or more processors, cause the one or more processors to: determine an analyte pattern type for each of a plurality of time of day (TOD) periods by executing a pattern analysis algorithm that receives as input time-correlated analyte data of a patient taken over an analysis period; select a recommended action based on the analyte pattern type; and store an indicator of the recommended action in a computer memory for output.

In some embodiments, the recommended action is a basal dosing recommendation.

In some embodiments, the instructions further cause the one or more processors to determine a measure of glucose dysregulation for the plurality of TOD periods, wherein the recommended action is selected based at least on the analyte pattern type and the measure of glucose dysregulation. In some embodiments, the measure of glucose dysregulation is determined by determining a number of times an analyte level crosses above or below a threshold crossing value in a time period. In some embodiments, the measure of glucose dysregulation is determined by determining a duration of time above or below a threshold value in a time period. In some embodiments, the measure of glucose dysregulation is determined by determining an area over or under a threshold area value in a time period. In some embodiments, the measure of glucose dysregulation is determined by determining a number of days in a time period with a minimum number of instances of glucose dysregulation.

In some embodiments, the instructions further cause the one or more processors to determine a frequency of a low glucose alarm within a time period, wherein the recommended action is selected based at least on the analyte pattern type and the frequency of the low glucose alarm. In some embodiments, the one or more processors determine the frequency of the low glucose alarm within the time period comprises determining if the low glucose alarm was triggered more than a threshold number of times.

In some embodiments, the instructions further cause the one or more processors to output the recommended action. In some embodiments, the recommended action is a change to a next dosing recommendation. In some embodiments, the change is a percentage of a current dose. In some embodiments, the change is a value of a dose in units.

In some embodiments, the recommended action is a recommendation to add a new medication. In some embodiments, the recommended action is outputted to an HCP. In some embodiments, the recommended action is outputted to a user. In some embodiments, the recommended action is an indication that a maximum recommended dose has been reached.

In some embodiments, the recommended action is an indication that an optimization in titration has been reached. In some embodiments, the recommended action further states that a user is in good glucose control. In some embodiments, the recommended action further states that a user remains in poor glucose control. In some embodiments, the recommended action further indicates that therapy escalation may be required.

In some embodiments, the instructions cause the one or more processors to select the recommended action based on the analyte pattern type and additional input. In some embodiments, the additional input comprises a user's weight. In some embodiments, the additional input comprises insulin dose data comprising dose amounts and corresponding times of administration. In some embodiments, the additional input comprises meal logs. In some embodiments, the additional input comprises exercise logs.

In many embodiments, a method for determining a titration for insulin dosing includes the steps of: determining, by at least one processor, a measure of glucose dysregulation for a plurality of time of day (TOD) periods by executing a pattern analysis algorithm that receives as input time-correlated analyte data of a patient taken over an analysis period; selecting, by the at least one processor executing a recommendation algorithm, a recommended action based on the measure of glucose dysregulation; and storing, by the at least one processor, an indicator of the recommended action in a computer memory for output.

In some embodiments, the measure of glucose dysregulation is determined by determining a number of times an analyte level crosses above or below a threshold crossing value in a time period.

In some embodiments, the measure of glucose dysregulation is determined by determining a duration of time above or below a threshold value in a time period.

In some embodiments, the measure of glucose dysregulation is determined by determining an area over or under a threshold area value in a time period.

In some embodiments, the measure of glucose dysregulation is determined by determining a number of days in a time period with a minimum number of instances of glucose dysregulation.

In some embodiments, the method further includes the step of determining, by at least one processor, an analyte pattern type for a plurality of time of day (TOD) periods by executing a pattern analysis algorithm that receives as input time-correlated analyte data of a patient taken over an analysis period, wherein the recommended action is selected based at least on the measure of glucose dysregulation and the determined analyte pattern.

In some embodiments, the method further includes the step of determining, by at least one processor, a frequency of a low glucose alarm within a time period, wherein the recommended action is selected based at least on the measure of glucose dysregulation and the frequency of the low glucose alarm.

In many embodiments, a system for determining a recommended medication dose includes an input configured to receive time-correlated analyte data of a patient taken over an analysis period; one or more processors coupled with the input and a memory storing instructions, wherein the instructions, when executed by the one or more processors, cause the one or more processors to: determine a measure of glucose dysregulation for a plurality of time of day (TOD) periods by executing a pattern analysis algorithm that receives as input time-correlated analyte data of a patient taken over an analysis period; select a recommended action based on the measure of glucose dysregulation; and store an indicator of the recommended action in a computer memory for output.

In some embodiments, the measure of glucose dysregulation is determined by determining a number of times an analyte level crosses above or below a threshold crossing value in a time period.

In some embodiments, the measure of glucose dysregulation is determined by determining a duration of time above or below a threshold value in a time period.

In some embodiments, the measure of glucose dysregulation is determined by determining an area over or under a threshold area value in a time period.

In some embodiments, the measure of glucose dysregulation is determined by determining a number of days in a time period with a minimum number of instances of glucose dysregulation.

In some embodiments, the instructions further cause the one or more processors to determine an analyte pattern type for a plurality of time of day (TOD) periods by executing a pattern analysis algorithm that receives as input time-correlated analyte data of a patient taken over an analysis period, wherein the recommended action is selected based at least on the measure of glucose dysregulation and the determined analyte pattern.

In some embodiments, the instructions further cause the one or more processors to determine a frequency of a low glucose alarm within a time period, wherein the recommended action is selected based at least on the measure of glucose dysregulation and the frequency of the low glucose alarm.

In many embodiments, a method for determining a titration for insulin dosing includes the steps of: determining, by at least one processor, a frequency of a low glucose alarm within a time period; selecting, by the at least one processor executing a recommendation algorithm, a recommended action based on the frequency of the low glucose alarm; and storing, by the at least one processor, an indicator of the recommended action in a computer memory for output.

In some embodiments, the step of determining, by the at least one processor, the frequency of the low glucose alarm within the time period comprises determining if the low glucose alarm was triggered more than a threshold number of times.

In some embodiments, the method further includes the step of outputting, by the at least one processor, the recommended action.

In some embodiments, the method further includes the step of determining, by at least one processor, an analyte pattern type for a plurality of time of day (TOD) periods by executing a pattern analysis algorithm that receives as input time-correlated analyte data of a patient taken over an analysis period, wherein the recommended action is selected based at least on the frequency of the low glucose alarm and the determined analyte pattern.

In some embodiments, the method further includes the step of determining, by at least one processor, a measure of glucose dysregulation for the plurality of TOD periods, wherein the recommended action is selected based at least on the frequency of the low glucose alarm and the measure of glucose dysregulation.

In many embodiments, a system for determining a recommended medication dose includes an input configured to receive time-correlated analyte data of a patient taken over an analysis period or a count of a number of low alarms over an analysis period; one or more processors coupled with the input and a memory storing instructions, wherein the instructions, when executed by the one or more processors, cause the one or more processors to: determine a frequency of a low glucose alarm within a time period; select a recommended action based on the frequency of the low glucose alarm; and store an indicator of the recommended action in a computer memory for output.

In some embodiments, the one or more processors determine the frequency of the low glucose alarm within the time period comprises determining if the low glucose alarm was triggered more than a threshold number of times.

In some embodiments, the instructions further cause the one or more processors to output the recommended action.

In some embodiments, the instructions further cause the one or more processors to determine an analyte pattern type for a plurality of time of day (TOD) periods by executing a pattern analysis algorithm that receives as input time-correlated analyte data of a patient taken over an analysis period, wherein the recommended action is selected based at least on the frequency of the low glucose alarm and the determined analyte pattern.

In some embodiments, the instructions further cause the one or more processors to determine a measure of glucose dysregulation for the plurality of TOD periods, wherein the recommended action is selected based at least on the frequency of the low glucose alarm and the measure of glucose dysregulation.

In many embodiments, a method for managing titration for insulin dosing includes the steps of determining, by at least one processor, a plurality of insulin dose recommendations based on a hypoglycemic risk analysis; determining, by the at least one processor, if no further titrations of an insulin dose should be recommended based on the plurality of insulin dose recommendations; and outputting, by the at least one processor, an indication that titration optimization has been reached.

In some embodiments, the step of determining if no further titrations of the insulin dose should be recommended comprises determining if a count of a same dose amount in the plurality of insulin dose recommendations is above a threshold value.

In some embodiments, the plurality of insulin dose recommendations comprises a portion of recommendations most recently outputted, wherein the step of determining if no further titrations of the insulin dose should be recommended comprises determining if the portion of recommendations most recently outputted does not trend in an upward or downward direction.

In some embodiments, the plurality of insulin dose recommendations comprises a portion of recommendations most recently outputted, wherein the step of determining if no further titrations of the insulin dose should be recommended comprises determining if amounts of consecutive doses in the portion of recommendations most recently outputted form an alternating pattern.

In some embodiments, the plurality of insulin dose recommendations are a plurality of basal insulin dose recommendations.

In some embodiments, the plurality of insulin dose recommendations are a plurality of bolus insulin dose recommendations.

In some embodiments, the hypoglycemic risk analysis comprises determining, by at least one processor, an analyte pattern type for each of a plurality of time of day (TOD) periods by executing a pattern analysis algorithm that receives as input time-correlated analyte data of a patient taken over an analysis period.

In some embodiments, the hypoglycemic risk analysis comprises determining, by at least one processor, a measure of glucose dysregulation for a plurality of time of day (TOD) periods by executing a pattern analysis algorithm that receives as input time-correlated analyte data of a patient taken over an analysis period.

In some embodiments, the hypoglycemic risk analysis comprises determining, by at least one processor, a frequency of a low glucose alarm within a time period.

In some embodiments, the hypoglycemic risk analysis comprises determining an analyte pattern type for each of a plurality of time of day (TOD) periods by executing a pattern analysis algorithm that receives as input time-correlated analyte data of a patient taken over an analysis period and at least one of determining a measure of glucose dysregulation and determining a frequency of a low glucose alarm within a time period.

In many embodiments, a system for managing titration for insulin dosing includes an input configured to receive dose data comprising data related to a plurality of doses administered during a period of time; one or more processors coupled with the input and a memory storing instructions, wherein the instructions, when executed by the one or more processors, cause the one or more processors to: determine a plurality of insulin dose recommendations based on a hypoglycemic risk analysis; determine if no further titrations of an insulin dose should be recommended based on the plurality of insulin dose recommendations; and output an indication that titration optimization has been reached.

In some embodiments, the one or more processors determine if no further titrations of the insulin dose should be recommended by determining if a count of a same dose amount in the plurality of insulin dose recommendations is above a threshold value.

In some embodiments, the plurality of insulin dose recommendations comprises a portion of recommendations most recently outputted, wherein the step of determining if no further titrations of the insulin dose should be recommended comprises determining if the portion of recommendations most recently outputted does not trend in an upward or downward direction.

In some embodiments, the plurality of insulin dose recommendations comprises a portion of recommendations most recently outputted, wherein the step of determining if no further titrations of the insulin dose should be recommended comprises determining if amounts of consecutive doses in the portion of recommendations most recently outputted form an alternating pattern.

In some embodiments, the plurality of insulin dose recommendations are a plurality of basal insulin dose recommendations.

In some embodiments, the plurality of insulin dose recommendations are a plurality of bolus insulin dose recommendations.

In some embodiments, the hypoglycemic risk analysis comprises determining, by at least one processor, an analyte pattern type for each of a plurality of time of day (TOD) periods by executing a pattern analysis algorithm that receives as input time-correlated analyte data of a patient taken over an analysis period.

In some embodiments, the hypoglycemic risk analysis comprises determining, by at least one processor, a measure of glucose dysregulation for a plurality of time of day (TOD) periods by executing a pattern analysis algorithm that receives as input time-correlated analyte data of a patient taken over an analysis period.

In some embodiments, the hypoglycemic risk analysis comprises determining, by at least one processor, a frequency of a low glucose alarm within a time period.

In some embodiments, the hypoglycemic risk analysis comprises determining an analyte pattern type for each of a plurality of time of day (TOD) periods by executing a pattern analysis algorithm that receives as input time-correlated analyte data of a patient taken over an analysis period and at least one of determining a measure of glucose dysregulation and determining a frequency of a low glucose alarm within a time period.

### CLAUSES

Exemplary embodiments are set out in the following numbered clauses.

Clause 1. A method for determining a titration for basal insulin dosing, the method comprising:
determining, by at least one processor, an analyte pattern type for each of a plurality of time of day (TOD) periods by executing a pattern analysis algorithm that receives as input time-correlated analyte data of a patient taken over an analysis period;
selecting, by the at least one processor executing a recommendation algorithm, a recommended action based on the analyte pattern type; and
storing, by the at least one processor, an indicator of the recommended action in a computer memory for output.

Clause 2. The method of clause 1, wherein the recommended action is a basal dosing recommendation.

Clause 3. The method of any of clauses 1-2, further comprising the step of:
determining, by at least one processor, a measure of glucose dysregulation for the plurality of TOD periods,
wherein the recommended action is selected based at least on the analyte pattern type and the measure of glucose dysregulation.

Clause 4. The method of any of clauses 1-3, wherein the measure of glucose dysregulation is determined by determining a number of times an analyte level crosses above or below a threshold crossing value in a time period.

Clause 5. The method of any of clauses 1-4, wherein the measure of glucose dysregulation is determined by determining a duration of time above or below a threshold value in a time period.

Clause 6. The method of any of clauses 1-5, wherein the measure of glucose dysregulation is determined by determining an area over or under a threshold area value in a time period.

Clause 7. The method of any of clauses 1-6, wherein the measure of glucose dysregulation is determined by determining a number of days in a time period with a minimum number of instances of glucose dysregulation.

Clause 8. The method of any of clauses 1-7, further comprising the step of:
determining, by at least one processor, a frequency of a low glucose alarm within a time period,
wherein the recommended action is selected based at least on the analyte pattern type and the frequency of the low glucose alarm.

Clause 9. The method of any of clauses 1-8, wherein the step of determining, by the at least one processor, the frequency of the low glucose alarm within the time period comprises determining if the low glucose alarm was triggered more than a threshold number of times.

Clause 10. The method of any of clauses 1-9, further comprising the step of:
outputting, by the at least one processor, the recommended action.

Clause 11. The method of any of clauses 1-10, wherein the recommended action is a change to a next dosing recommendation.

Clause 12. The method of any of clauses 1-11, wherein the change is a percentage of a current dose.

Clause 13. The method of any of clauses 1-12, wherein the change is a value of a dose in units.

Clause 14. The method of any of clauses 1-13, wherein the recommended action is a recommendation to add a new medication.

Clause 15. The method of any of clauses 1-14, wherein the recommended action is outputted to an HCP.

Clause 16. The method of any of clauses 1-15, wherein the recommended action is outputted to a user.

Clause 17. The method of any of clauses 1-16, wherein the recommended action is an indication that a maximum recommended dose has been reached.

Clause 18. The method of any of clauses 1-17, wherein the recommended action is an indication that an optimization in titration has been reached.

Clause 19. The method of any of clauses 1-18, wherein the recommended action further states that a user is in good glucose control.

Clause 20. The method of any of clauses 1-19, wherein the recommended action further states that a user remains in poor glucose control.

Clause 21. The method of any of clauses 1-20, wherein the recommended action further indicates that therapy escalation may be required.

Clause 22. The method of any of clauses 1-21, wherein selecting the recommended action is based on the analyte pattern type and additional input.

Clause 23. The method of any of clauses 1-22, wherein the additional input comprises a user's weight.

Clause 24. The method of any of clauses 1-23, wherein the additional input comprises insulin dose data comprising dose amounts and corresponding times of administration.

Clause 25. The method of any of clauses 1-24, wherein the additional input comprises meal logs.

Clause 26. The method of any of clauses 1-25, wherein the additional input comprises exercise logs.

Clause 27. A system for determining a recommended medication dose, the system comprising:
an input configured to receive time-correlated analyte data of a patient taken over an analysis period;
   one or more processors coupled with the input and a memory storing instructions, wherein the instructions, when executed by the one or more processors, cause the one or more processors to:
      determine an analyte pattern type for each of a plurality of time of day (TOD) periods by executing a pattern analysis algorithm that receives as input time-correlated analyte data of a patient taken over an analysis period;
         select a recommended action based on the analyte pattern type; and
         store an indicator of the recommended action in a computer memory for output.

Clause 28. The system of clause 27, wherein the recommended action is a basal dosing recommendation.

Clause 29. The system of any of clauses 27-28, wherein the instructions further cause the one or more processors to:
determine a measure of glucose dysregulation for the plurality of TOD periods,
wherein the recommended action is selected based at least on the analyte pattern type and the measure of glucose dysregulation.

Clause 30. The system of any of clauses 27-29, wherein the measure of glucose dysregulation is determined by determining a number of times an analyte level crosses above or below a threshold crossing value in a time period.

Clause 31. The system of any of clauses 27-30, wherein the measure of glucose dysregulation is determined by determining a duration of time above or below a threshold value in a time period.

Clause 32. The system of any of clauses 27-31, wherein the measure of glucose dysregulation is determined by determining an area over or under a threshold area value in a time period.

Clause 33. The system of any of clauses 27-32, wherein the measure of glucose dysregulation is determined by determining a number of days in a time period with a minimum number of instances of glucose dysregulation.

Clause 34. The system of any of clauses 27-33, wherein the instructions further cause the one or more processors to:
determine a frequency of a low glucose alarm within a time period,
wherein the recommended action is selected based at least on the analyte pattern type and the frequency of the low glucose alarm.

Clause 35. The system of any of clauses 27-34, wherein the one or more processors determine the frequency of the low glucose alarm within the time period comprises determining if the low glucose alarm was triggered more than a threshold number of times.

Clause 36. The system of any of clauses 27-35, wherein the instructions further cause the one or more processors to output the recommended action.

Clause 37. The system of any of clauses 27-36, wherein the recommended action is a change to a next dosing recommendation.

Clause 38. The system of any of clauses 27-37, wherein the change is a percentage of a current dose.

Clause 39. The system of any of clauses 27-38, wherein the change is a value of a dose in units.

Clause 40. The system of any of clauses 27-39, wherein the recommended action is a recommendation to add a new medication.

Clause 41. The system of any of clauses 27-40, wherein the recommended action is outputted to an HCP.

Clause 42. The system of any of clauses 27-41, wherein the recommended action is outputted to a user.

Clause 43. The system of any of clauses 27-42, wherein the recommended action is an indication that a maximum recommended dose has been reached.

Clause 44. The system of any of clauses 27-43, wherein the recommended action is an indication that an optimization in titration has been reached.

Clause 45. The system of clauses 27-44, wherein the recommended action further states that a user is in good glucose control.

Clause 46. The system of clauses 27-45, wherein the recommended action further states that a user remains in poor glucose control.

Clause 47. The system of clauses 27-46, wherein the recommended action further indicates that therapy escalation may be required.

Clause 48. The system of clauses 27-47, wherein the instructions cause the one or more processors to select the recommended action based on the analyte pattern type and additional input.

Clause 49. The system of clauses 27-48, wherein the additional input comprises a user's weight.

Clause 50. The system of clauses 27-49, wherein the additional input comprises insulin dose data comprising dose amounts and corresponding times of administration.

Clause 51. The system of clauses 27-50, wherein the additional input comprises meal logs.

Clause 52. The system of clauses 27-51, wherein the additional input comprises exercise logs.

Clause 53. A method for determining a titration for insulin dosing, the method comprising:
determining, by at least one processor, a measure of glucose dysregulation for a plurality of time of day (TOD) periods by executing a pattern analysis algorithm that receives as input time-correlated analyte data of a patient taken over an analysis period;
selecting, by the at least one processor executing a recommendation algorithm, a recommended action based on the measure of glucose dysregulation; and
storing, by the at least one processor, an indicator of the recommended action in a computer memory for output.

Clause 54. The method of clause 53, wherein the measure of glucose dysregulation is determined by determining a number of times an analyte level crosses above or below a threshold crossing value in a time period.

Clause 55. The method of any of clauses 53-54, wherein the measure of glucose dysregulation is determined by determining a duration of time above or below a threshold value in a time period.

Clause 56. The method of any of clauses 53-55, wherein the measure of glucose dysregulation is determined by determining an area over or under a threshold area value in a time period.

Clause 57. The method of any of clauses 53-56, wherein the measure of glucose dysregulation is determined by determining a number of days in a time period with a minimum number of instances of glucose dysregulation.

Clause 58. The method of any of clauses 53-57, further comprising the step of:
determining, by at least one processor, an analyte pattern type for a plurality of time of day (TOD) periods by executing a pattern analysis algorithm that receives as input time-correlated analyte data of a patient taken over an analysis period,
wherein the recommended action is selected based at least on the measure of glucose dysregulation and the determined analyte pattern.

Clause 59. The method of any of clauses 53-58, further comprising the step of:
determining, by at least one processor, a frequency of a low glucose alarm within a time period,
wherein the recommended action is selected based at least on the measure of glucose dysregulation and the frequency of the low glucose alarm.

Clause 60. A system for determining a recommended medication dose, the system comprising:
an input configured to receive time-correlated analyte data of a patient taken over an analysis period;
   one or more processors coupled with the input and a memory storing instructions, wherein the instructions, when executed by the one or more processors, cause the one or more processors to:
      determine a measure of glucose dysregulation for a plurality of time of day (TOD) periods by executing a pattern analysis algorithm that receives as input time-correlated analyte data of a patient taken over an analysis period;
         select a recommended action based on the measure of glucose dysregulation; and
         store an indicator of the recommended action in a computer memory for output.

Clause 61. The system of clause 60, wherein the measure of glucose dysregulation is determined by determining a number of times an analyte level crosses above or below a threshold crossing value in a time period.

Clause 62. The system of any of clauses 60-61, wherein the measure of glucose dysregulation is determined by determining a duration of time above or below a threshold value in a time period.

Clause 63. The system of any of clauses 60-62, wherein the measure of glucose dysregulation is determined by determining an area over or under a threshold area value in a time period.

Clause 64. The system of any of clauses 60-63, wherein the measure of glucose dysregulation is determined by determining a number of days in a time period with a minimum number of instances of glucose dysregulation.

Clause 65. The system of any of clauses 60-64, wherein the instructions further cause the one or more processors to:
determine an analyte pattern type for a plurality of time of day (TOD) periods by executing a pattern analysis algorithm that receives as input time-correlated analyte data of a patient taken over an analysis period,
wherein the recommended action is selected based at least on the measure of glucose dysregulation and the determined analyte pattern.

Clause 66. The system of any of clauses 60-65, wherein the instructions further cause the one or more processors to:
determine a frequency of a low glucose alarm within a time period,
wherein the recommended action is selected based at least on the measure of glucose dysregulation and the frequency of the low glucose alarm.

Clause 67. A method for determining a titration for insulin dosing, the method comprising:
determining, by at least one processor, a frequency of a low glucose alarm within a time period;
selecting, by the at least one processor executing a recommendation algorithm, a recommended action based on the frequency of the low glucose alarm; and
storing, by the at least one processor, an indicator of the recommended action in a computer memory for output.

Clause 68. The method of clause 67, wherein the step of determining, by the at least one processor, the frequency of the low glucose alarm within the time period comprises determining if the low glucose alarm was triggered more than a threshold number of times.

Clause 69. The method of any of clauses 67-68, further comprising the step of:
outputting, by the at least one processor, the recommended action.

Clause 70. The method of any of clauses 67-69, further comprising the step of:
determining, by at least one processor, an analyte pattern type for a plurality of time of day (TOD) periods by executing a pattern analysis algorithm that receives as input time-correlated analyte data of a patient taken over an analysis period,
wherein the recommended action is selected based at least on the frequency of the low glucose alarm and the determined analyte pattern.

Clause 71. The method of any of clauses 67-70, further comprising the step of:
determining, by at least one processor, a measure of glucose dysregulation for the plurality of TOD periods,
wherein the recommended action is selected based at least on the frequency of the low glucose alarm and the measure of glucose dysregulation.

Clause 72. A system for determining a recommended medication dose, the system comprising:
an input configured to receive time-correlated analyte data of a patient taken over an analysis period or a count of a number of low alarms over an analysis period;
   one or more processors coupled with the input and a memory storing instructions, wherein the instructions, when executed by the one or more processors, cause the one or more processors to:
      determine a frequency of a low glucose alarm within a time period;
         select a recommended action based on the frequency of the low glucose alarm; and
         store an indicator of the recommended action in a computer memory for output.

Clause 73. The system of clause 72, wherein the one or more processors determine the frequency of the low glucose alarm within the time period comprises determining if the low glucose alarm was triggered more than a threshold number of times.

Clause 74. The system of any of clauses 72-73, wherein the instructions further cause the one or more processors to output the recommended action.

Clause 75. The system of any of clauses 72-74, wherein the instructions further cause the one or more processors to:
determine an analyte pattern type for a plurality of time of day (TOD) periods by executing a pattern analysis algorithm that receives as input time-correlated analyte data of a patient taken over an analysis period,
wherein the recommended action is selected based at least on the frequency of the low glucose alarm and the determined analyte pattern.

Clause 76. The system of any of clauses 72-75, wherein the instructions further cause the one or more processors to:
determine a measure of glucose dysregulation for the plurality of TOD periods,
wherein the recommended action is selected based at least on the frequency of the low glucose alarm and the measure of glucose dysregulation.

Clause 77. A method for managing titration for insulin dosing, the method comprising:
determining, by at least one processor, a plurality of insulin dose recommendations based on a hypoglycemic risk analysis;
determining, by the at least one processor, if no further titrations of an insulin dose should be recommended based on the plurality of insulin dose recommendations; and outputting, by the at least one processor, an indication that titration optimization has been reached.

Clause 78. The method of clause 77, wherein the step of determining if no further titrations of the insulin dose should be recommended comprises determining if a count of a same dose amount in the plurality of insulin dose recommendations is above a threshold value.

Clause 79. The method of any of clauses 77-78, wherein the plurality of insulin dose recommendations comprises a portion of recommendations most recently outputted, wherein the step of determining if no further titrations of the insulin dose should be recommended comprises determining if the portion of recommendations most recently outputted does not trend in an upward or downward direction.

Clause 80. The method of any of clauses 77-79, wherein the plurality of insulin dose recommendations comprises a portion of recommendations most recently outputted, wherein the step of determining if no further titrations of the insulin dose should be recommended comprises determining if amounts of consecutive doses in the portion of recommendations most recently outputted form an alternating pattern.

Clause 81. The method of any of clauses 77-80, wherein the plurality of insulin dose recommendations are a plurality of basal insulin dose recommendations.

Clause 82. The method of any of clauses 77-81, wherein the plurality of insulin dose recommendations are a plurality of bolus insulin dose recommendations.

Clause 83. The method of any of clauses 77-82, wherein the hypoglycemic risk analysis comprises determining, by at least one processor, an analyte pattern type for each of a plurality of time of day (TOD) periods by executing a pattern analysis algorithm that receives as input time-correlated analyte data of a patient taken over an analysis period.

Clause 84. The method of any of clauses 77-83, wherein the hypoglycemic risk analysis comprises determining, by at least one processor, a measure of glucose dysregulation for a plurality of time of day (TOD) periods by executing a pattern analysis algorithm that receives as input time-correlated analyte data of a patient taken over an analysis period.

Clause 85. The method of any of clauses 77-84, wherein the hypoglycemic risk analysis comprises determining, by at least one processor, a frequency of a low glucose alarm within a time period.

Clause 86. The method of any of clauses 77-85, wherein the hypoglycemic risk analysis comprises determining an analyte pattern type for each of a plurality of time of day (TOD) periods by executing a pattern analysis algorithm that receives as input time-correlated analyte data of a patient taken over an analysis period and at least one of determining a measure of glucose dysregulation and determining a frequency of a low glucose alarm within a time period.

Clause 87. A system for managing titration for insulin dosing, the system comprising:
an input configured to receive dose data comprising data related to a plurality of doses administered during a period of time;
   one or more processors coupled with the input and a memory storing instructions, wherein the instructions, when executed by the one or more processors, cause the one or more processors to:
      determine a plurality of insulin dose recommendations based on a hypoglycemic risk analysis;
         determine if no further titrations of an insulin dose should be recommended based on the plurality of insulin dose recommendations; and
         output an indication that titration optimization has been reached.

Clause 88. The system of clause 87, wherein the one or more processors determine if no further titrations of the insulin dose should be recommended by determining if a count of a same dose amount in the plurality of insulin dose recommendations is above a threshold value.

Clause 89. The system of any of clauses 87-88, wherein the plurality of insulin dose recommendations comprises a portion of recommendations most recently outputted, wherein the step of determining if no further titrations of the insulin dose should be recommended comprises determining if the portion of recommendations most recently outputted does not trend in an upward or downward direction.

Clause 90. The system of any of clauses 87-89, wherein the plurality of insulin dose recommendations comprises a portion of recommendations most recently outputted, wherein the step of determining if no further titrations of the insulin dose should be recommended comprises determining if amounts of consecutive doses in the portion of recommendations most recently outputted form an alternating pattern.

Clause 91. The system of any of clauses 87-90, wherein the plurality of insulin dose recommendations are a plurality of basal insulin dose recommendations.

Clause 92. The system of any of clauses 87-91, wherein the plurality of insulin dose recommendations are a plurality of bolus insulin dose recommendations.

Clause 93. The system of any of clauses 87-92, wherein the hypoglycemic risk analysis comprises determining, by at least one processor, an analyte pattern type for each of a plurality of time of day (TOD) periods by executing a pattern analysis algorithm that receives as input time-correlated analyte data of a patient taken over an analysis period.

Clause 94. The system of any of clauses 87-93, wherein the hypoglycemic risk analysis comprises determining, by at least one processor, a measure of glucose dysregulation for a plurality of time of day (TOD) periods by executing a pattern analysis algorithm that receives as input time-correlated analyte data of a patient taken over an analysis period.

Clause 95. The system of any of clauses 87-94, wherein the hypoglycemic risk analysis comprises determining, by at least one processor, a frequency of a low glucose alarm within a time period.

Clause 96. The system of any of clauses 87-95, wherein the hypoglycemic risk analysis comprises determining an analyte pattern type for each of a plurality of time of day (TOD) periods by executing a pattern analysis algorithm that receives as input time-correlated analyte data of a patient taken over an analysis period and at least one of determining a measure of glucose dysregulation and determining a frequency of a low glucose alarm within a time period.

1. A system for determining a recommended medication dose, the system comprising:
an input configured to receive time-correlated analyte data of a patient taken over an analysis period;
one or more processors coupled with the input and a memory storing instructions, wherein the instructions, when executed by the one or more processors, cause the one or more processors to:
determine an analyte pattern type for each of a plurality of time of day (TOD) periods by executing a pattern analysis algorithm that receives as input time-correlated analyte data of a patient taken over an analysis period;
select a recommended action based on the analyte pattern type; and
store an indicator of the recommended action in a computer memory for output.

2. The system of claim 1, wherein the recommended action is a basal dosing recommendation.

3. The system of claim 1, wherein the instructions further cause the one or more processors to:
determine a measure of glucose dysregulation for the plurality of TOD periods,
wherein the recommended action is selected based at least on the analyte pattern type and the measure of glucose dysregulation.

4. The system of claim 3, wherein the measure of glucose dysregulation is determined by determining a number of times an analyte level crosses above or below a threshold crossing value in a time period.

5. The system of claim 3, wherein the measure of glucose dysregulation is determined by determining a duration of time above or below a threshold value in a time period.

6. The system of claim 3, wherein the measure of glucose dysregulation is determined by determining an area over or under a threshold area value in a time period.

7. The system of claim 3, wherein the measure of glucose dysregulation is determined by determining a number of days in a time period with a minimum number of instances of glucose dysregulation.

8. The system of claim 1, wherein the instructions further cause the one or more processors to:
determine a frequency of a low glucose alarm within a time period,
wherein the recommended action is selected based at least on the analyte pattern type and the frequency of the low glucose alarm.

9. (canceled)

10. The system of claim 1, wherein the instructions further cause the one or more processors to output the recommended action.

11. The system of claim 10, wherein the recommended action is a change to a next dosing recommendation.

12. (canceled)

13. (canceled)

14. The system of claim 10, wherein the recommended action is a recommendation to add a new medication.

15. (canceled)

16. (canceled)

17. The system of claim 10, wherein the recommended action is an indication that a maximum recommended dose has been reached.

18. The system of claim 10, wherein the recommended action is an indication that an optimization in titration has been reached.

19. (canceled)

20. The system of claim 18, wherein the recommended action further states that a user remains in poor glucose control.

21. The system of claim 20, wherein the recommended action further indicates that therapy escalation may be required.

22. The system of claim 2, wherein the instructions cause the one or more processors to select the recommended action based on the analyte pattern type and additional input.

23. The system of claim 22, wherein the additional input comprises a user's weight.

24. The system of claim 22, wherein the additional input comprises insulin dose data comprising dose amounts and corresponding times of administration.

25. The system of claim 22, wherein the additional input comprises meal logs.

26. The system of claim 22, wherein the additional input comprises exercise logs.

27-96. (canceled)
